(19) United States
(12) Patent Application Publication
(10) Pub. No.: US 2005/0132505 A9
(43) Pub. Date: Jun. 23, 2005

(54) Bleaching composition for dyed keratinous fibers

(76) Inventors: Frederic Legrand, Boulogne Billancourt (FR); Jean-Marie Millequant, Saint Maur des Fosses (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/362,077

(22) PCT Filed: Aug. 14, 2001
(86) PCT No.: PCT/FR01/02612

(30) Foreign Application Priority Data
Aug. 21, 2000 (FR) 00/10756

(51) Int. Cl. A61K 7/13

(52) U.S. Cl. 8/405

(57) ABSTRACT

The invention concerns a composition for bleaching keratinous fibres dyed with oxidation dyes and/or direct dyes, in particular human keratinous fibres such as hair, comprising in a suitable bleaching medium with acid pH, a combination based on at least a sulphinic acid and at least an alpha-oxocarboxylic acid and/or their respective cosmetically acceptable salts. The invention also concerns multi-part kits devices for dyeing and bleaching said fibres and the bleaching method using said composition.

A subject matter of the invention is a composition for bleaching keratinous fibers dyed with oxidation dyes and/or direct dyes, in particular human keratinous fibers, such as the hair, comprising, in a medium appropriate for rinsing at an acidic pH, a combination based on at least one sulfinic acid and at least one α-oxocarboxylic acid and/or their respective cosmetically acceptable salts. The invention also relates to multicompartment devices for dyeing and bleaching said fibers and to the bleaching process employing this composition.

It is known to dye keratinous fibers and in particular human hair with dyeing compositions comprising oxidation dyes and/or direct dyes.

The dyeing carried out with oxidation dyes or “oxidation coloring” is a permanent coloring; it comprises, as oxidation dyes, oxidation coloring precursors and couplers.

Oxidation coloring precursors, commonly known as “oxidation bases”, are initially colorless or weakly colored compounds which develop their dyeing power within the individual hair in the presence of oxidizing agents added at the time of use, leading to the formation of colored and coloring compounds. The formation of these colored and coloring compounds results either from an oxidative coupling of “oxidation bases” with one another or from an oxidative coupling of the “oxidation bases” with coloration-modifying compounds, commonly known as “couplers”, generally present in the dyeing compositions used in oxidation dyeing.

The oxidation bases are in particular ortho- or para-phenylenediamines, ortho- or para-aminophenols, and heterocyclic bases.

The couplers are chosen in particular from aromatic meta-diamines, meta-aminophenols, meta-diphenols and certain heterocyclic compounds.

The variety of the molecules involved in the oxidation bases and couplers makes it possible to obtain a rich palette of colors.

To vary the shades obtained with said oxidation dyes or to enrich them with highlights, direct dyes may be added to them.

The dyeing carried out with direct dyes gives a semipermanent or temporary coloring; direct dyes give a more or less marked modification in color to the natural coloring of the hair which may possibly withstand being shampooed several times.

These direct dyes can be employed without an oxidizing agent.

The direct dyes conventionally used are chosen in particular from nitrobenzene direct dyes, azo direct dyes, quinone direct dyes and in particular anthroquinone direct dyes, azine direct dyes, triarylmethane direct dyes, indoamine direct dyes and natural direct dyes.

More recently, in patent applications WO 95/15144, WO 95/01772 and EP-1 025 834, the use has been recommended of novel cationic direct dyes which are distinguished from conventional direct dyes by the highly chromatic shades which they confer on keratinous fibers.

Said cationic direct dyes can be used in an oxidizing medium.

In the presence of an oxidizing agent, the aim is to obtain a lightening coloring. Lightening coloring is carried out by applying, to the hair, the mixture of the cationic direct dye and of an oxidizing agent which is prepared at the time of use and makes it possible in particular to obtain an advantageous effect, such as a uniform color in the case of gray hair or of bringing out the color in the case of naturally pigmented hair, by lightening the melanin of the hair.

However, for various reasons, such as the wish to partially or completely modify the shade thus conferred on the hair by an oxidation dyeing, a direct dyeing or a lightening dyeing or the wish to remove this coloring, a desire may be induced to partially or completely destroy the pigments thus formed in or on the individual hair.

This bleaching has, until now, been carried out via processes employing oxidizing or reducing systems.

In oxidizing systems, the oxidizing agents conventionally used are hydrogen peroxide or compounds capable of producing hydrogen peroxide by hydrolysis, such as urea hydrogen peroxide, or persalts, such as perborates, percarbonates and persulfates, hydrogen peroxide and persulfates being particularly preferred.

Among reducing systems, the use of hydroxymethanesulfinic acid at a pH of between 7 and 9 for bleaching colored hair is known from German patent No. 1 151 242.

A process for bleaching dyed keratinous fibers is also disclosed in patent U.S. Pat. No. 3,892,845 and consists in applying, to the fibers, an aqueous composition comprising the combination of two types of reducing agents, an agent which reduces the dye and an agent which reduces the disulfide covalent bonds of the keratin; the agent which reduces the dye is a zinc, potassium, sodium or calcium hydroxymethanesulfinate or hydrosulfite and the agent which reduces the keratin is in particular thioglycolic acid, a potassium or sodium bisulfate or bisulfite, potassium disulfide, thiourea or certain phosphorus compounds.

However, it is also known to bleach human keratinous fibers, such as the hair and in particular hair artificially dyed with oxidation dyes, using reducing agents at an acidic pH.

The technology of bleaching by reduction at an acidic pH has the advantage of sensitizing the hair fiber to a lesser extent and of not lightening the natural base of the hair.

Thus, commercial products use sodium hydroxymethanesulfinate as reducing agent for dyed hair at an acidic pH.

And, in patent application EP-0 943 316, the use has recently been recommended of a combination, at an acidic pH, comprising ascorbic acid and α-oxocarboxylic acid for bleaching human hair dyed beforehand with an oxidation dye, and a commercial product for bleaching the hair comprises ascorbic acid and α-ketoglutaric acid.

However, the Applicant Company has found that none of these techniques of the prior art produces a sufficiently effective bleaching of keratinous fibers dyed with conventional direct dyes and/or oxidation dyes.

This is because the bleaching is only slightly effective, indeed even ineffective, with respect to certain shades, such as basic hues and shades comprising gold and ash highlights.

In addition, this type of bleaching does not make it possible to sufficiently strip keratinous fibers dyed with highly chromatic cationic direct dyes, such as in particular those recently disclosed in patent applications WO 95/15144, WO 95/01772 and EP-1 025 834.

The Applicant Company has now just discovered, unexpectedly and entirely surprisingly, that it has become possible to very significantly improve the bleaching of keratinous fibers dyed beforehand with conventional direct dyes and/or oxidation dyes and to allow the removal of a much broader palette of colors.

More particularly still, it has now become possible to partially or completely bleach keratinous fibers dyed with cationic direct dyes, such as those described above.

These discoveries therefore form the basis of the present invention.

A first subject matter of the invention is thus a composition for bleaching keratinous fibers dyed with oxidation dyes and/or direct dyes, in particular human keratinous fibers, such as the hair, comprising at least one dye reducing agent in an aqueous medium appropriate for bleaching at an acidic pH and characterized in that said reducing agent is a system combining (i) at least one sulfinic acid or one of their cosmetically acceptable salts with (ii) at least one α-oxocarboxylic acid or one of their cosmetically acceptable salts.

Said bleaching can be partial or complete.

The Applicant Company has in particular been able to find, very surprisingly, that said system is synergistic in bleaching.

Compositions intended for bleaching the hair using reducing agents are mainly provided in the form of ready-to-use compositions composed of anhydrous products (powders) or of creams or of gels comprising the reducing agent or agents which are mixed at the time of use with an aqueous composition comprising a pH agent. Bleaching compositions are also provided in the form of aqueous ready-to-use compositions comprising the reducing agent or agents at the appropriate pH.

The composition according to the invention is a ready-to-use composition.

And the term “ready-to-use composition” is to be understood, within the meaning of the invention, as the composition intended to be applied as such to keratinous fibers, that is to say that it can be stored as such before use or can result from the mixing at the time of use of two or more compositions.

According to the present invention, preference is given, among sulfinic acids, to the use of hydroxymethanesulfinic acid.

The cosmetically acceptable salts of the sulfinic acids according to the invention can be chosen from alkali metal (Na, K) sulfinates, alkaline earth metal (Ca) sulfinates or zinc sulfinates.

Sodium hydroxymethanesulfinate is more particularly preferred according to the invention.

The sulfinic acid or acids or their salts can be present in the composition in proportions of between 0.01 and 20% and preferably between 0.1 and 10% by weight of the total weight of the composition.

According to the present invention, the α-oxocarboxylic acid can be chosen from oxalic acid, glyoxalic acid, pyruvic acid and, preferably, α-ketoglutaric acid.

The cosmetically acceptable salts of these acids are in particular, for example, alkali metal or alkaline earth metal salts.

The α-oxocarboxylic acid or acids or their salts can be present in the composition in proportions of between 0.01 and 15% and preferably between 0.1 and 10% by weight of the total weight of the composition.

Care will preferably be taken that the ratio by weight of the sulfinic acid or acids or their salts to the α-oxocarboxylic acid or acids or their salts is between 10/1 and 1/10.

Another subject matter of the invention is multicompartment devices or packaging kits intended (i) for the dyeing and then (ii) for the bleaching of keratinous fibers dyed with oxidation dyes and/or direct dyes, in particular human keratinous fibers, such as the hair, characterized in that they comprise a first compartment comprising a composition for the oxidizing dyeing or the nonoxidizing dyeing of said fibers and, in a second compartment, a composition for the reductive bleaching of said colored fibers comprising the bleaching system described above at an acidic pH.

The invention is also targeted at a process for bleaching keratinous fibers dyed with oxidation dyes and/or direct dyes and in particular human keratinous fibers, such as the hair, employing a bleaching composition as described above.

In the first compartment of the kit, the oxidizing dye can be a conventional oxidation dye, a direct dye or a lightening dye.

In the case of a conventional oxidation dye, the fibers are dyed beforehand with at least one oxidation dye and preferably with at least one oxidation base in the presence of an oxidizing agent.

Thus, in the first compartment of the kit where it is recommended to use an oxidizing dye, such as the conventional oxidation dye, there will be found at least one oxidation base preferably chosen from para-phenylenediamine and its derivatives substituted on one of the amine functional groups and/or on the benzene ring, para-aminophenol and its derivatives substituted on the amine functional groups and/or on the benzene ring, double bases, ortho-aminophenols, ortho-phenylenediamines and heterocyclic bases.

Mention may more particularly be made, among heterocyclic bases which can be used as oxidation base according to the invention, of pyridine derivatives, pyrimidine derivatives or pyrazole derivatives. All these compounds can be used in the free form or in the form of their addition salts with an acid.

Mention may in particular be made of:
(I) the para-phenylenediamines of following formula (I) and their addition salts with an acid: 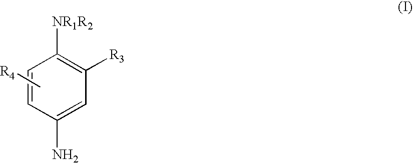
   in which:
R₁ represents a hydrogen atom, a C₁-C₄ alkyl radical, a C₁-C₄ monohydroxyalkyl radical, a C₂-C₄ polyhydroxyalkyl radical, a (C₁-C₄)alkoxy(C₁-C₄)alkyl radical or a C₁-C₄ alkyl radical substituted by a nitrogenous, phenyl or 4′-aminophenyl group;
R₂ represents a hydrogen atom, a C₁-C₄ alkyl radical, a C₁-C₄ monohydroxyalkyl radical, a C₂-C₄ polyhydroxyalkyl radical, a (C₁-C₄)alkoxy(C₁-C₄)alkyl radical or a C₁-C₄ alkyl radical substituted by a nitrogenous group;
R₃ represents a hydrogen atom, a halogen atom, such as a chlorine atom, a C₁-C₄ alkyl radical, a sulfo radical, a carboxyl radical, a C₁-C₄ monohydroxyalkyl radical, a C₁-C₄ hydroxyalkoxy radical, a C₁-C₄ acetylaminoalkoxy radical, a C₁-C₄ mesylaminoalkoxy radical or a C₁-C₄ carbamoylaminoalkoxy radical,
R₄ represents a hydrogen atom, a halogen atom or a C₁-C₄ alkyl radical;
R₁ and R₂ can also form, with the nitrogen atom which carries them, a 5- or 6-membered nitrogenous heterocycle optionally substituted by one or more alkyl, hydroxyl or ureido groups.

Mention may in particular be made, among the nitrogenous groups of the above formula (I), of amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Mention may more particularly be made, among the para-phenylenediamines of above formula (I), of para-phenylenediamine, para-toluylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl, β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 2-methyl-1-N-β-hydroxyethyl-para-phenylenediamine, and their addition salts with an acid.

Preference is very particularly given, among the para-phenylenediamines of above formula (I), to para-phenylenediamine, para-toluylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, and their addition salts with an acid.
(II) According to the invention, the term “double bases” is understood to mean compounds comprising at least two aromatic rings on which are carried amino and/or hydroxyl groups.

Mention may in particular be made, among the double bases which can be used as oxidation bases in the dyeing compositions in accordance with the invention, of the compounds corresponding to the following formula (II) and their addition salts with an acid: 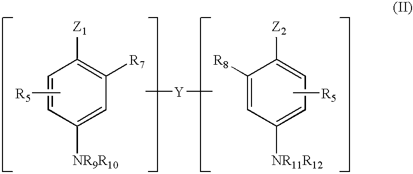
in which;
Z₁ and Z₂, which are identical or different, represent a hydroxyl or —NH₂ radical which can be substituted by a C₁-C₄ alkyl radical or by a connecting arm Y;
the connecting arm Y represents a linear or branched alkylene chain comprising from 1 to 14 carbon atoms which can be interrupted or terminated by one or more nitrogenous groups and/or by one or more heteroatoms, such as oxygen, sulfur or nitrogen atoms, and which is optionally substituted by one or more hydroxyl or C₁-C₆ alkoxy radicals;
R₅ and R₆ represent a hydrogen atom, a halogen atom, a C₁-C₄ alkyl radical, a C₁-C₄ monohydroxyalkyl radical, a C₂-C₄ polyhydroxyalkyl radical, a C₁-C₄ aminoalkyl radical or a connecting arm Y;
R₇, R₈, R₉, R₁₀, R₁₁ and R₁₂, which are identical or different, represent a hydrogen atom, a connecting arm Y or a C₁-C₄ alkyl radical;
it being understood that the compounds of formula (II) comprise only a single connecting arm Y per molecule.

Mention may in particular be made, among the nitrogenous groups of the above formula (II), of amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Mention may more particularly be made, among the double bases of above formulae (II), of N,N′-bis(p-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino, 3′-methylphenyl)ethylenediamine, 1,8-bis(2,5-diaminophenoxy)-3,5-dioxaoctane, and their addition salts with an acid.

Among these double bases of formula (II), N,N′-bis(β-hydroxyethyl)-N,N′-bis(4′-aminophenyl)-1,3-diaminopropanol, 1,8-bis(2,5-diaminophenoxy)3,5-dioxaoctane or one of their addition salts with an acid are particularly preferred.
(III) the para-aminophenols corresponding to the following formula (III) and their addition salts with an acid: 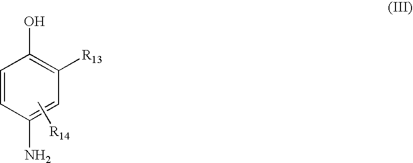
   in which:
R₁₃ represents a hydrogen atom, a halogen atom, such as fluorine, a C₁-C₄ alkyl radical, a C₁-C₄ monohydroxyalkyl radical, a (C₁-C₄)alkoxy(C₁-C₄)alkyl radical, a C₁-C₄aminoalkyl radical or a hydroxy(C₁-C₄)alkylamino(C₁-C₄)alkyl radical;
R₁₄ represents a hydrogen atom, a halogen atom, such as fluorine, a C₁-C₄ alkyl radical, a C₁-C₄ monohydroxyalkyl radical, a C₂-C₄ polyhydroxyalkyl radical, a C₁-C₄ aminoalkyl radical, a C₁-C₄ cyanoalkyl radical or a (C₁-C₄)alkoxy(C₁-C₄)alkyl radical.

Mention may more particularly be made, among the para-aminophenols of above formula (III), of para-aminophenol, 4-amino-3-methylphenol, 4-amino3-fluorophenol, 4-amino-3-(hydroxymethyl)phenol, 4-amino-2-methylphenol, 4amino-2-(hydroxymethyl)phenol, 4-amino-2-(methoxymethyl)phenol, 4-amino-2(aminomethyl)phenol, 4-amino-2-[(β-hydroxyethyl)aminomethyl]phenol, and their addition salts with an acid.
(IV) the ortho-aminophenols which can be used as oxidation bases in the context of the present invention are chosen in particular from 2-aminophenol, 2-amino-1-hyrdoxy-5-methylbenzene, 2-amino-1-hydroxy-6-methylbenzene, 5-acetamido-2aminophenol, and their addition salts with an acid.
(V) mention may more particularly be made, among the heterocyclic bases which can be used as oxidation bases in the dyeing compositions in accordance with the invention, of the pyridine derivatives, the pyrimidine derivatives, the pyrazole derivatives, and their addition salts with an acid.

Mention may more particularly be made, among the pyridine derivatives, of the compounds disclosed, for example, in patents GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino6-methoxypyridine, 3,4-diaminopyridine, and their addition salts with an acid.

Mention may more particularly be made, among the pyrimidine derivatives, of the compounds disclosed, for example, in German patent DE 2 359 399 or Japanese patents JP 88-169 571 and JP 91-10659 or patent applications WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and pyrazolopyrimidine derivatives, such as those mentioned in patent application FR-A-2 750 048 and among which may be mentioned pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5a]pyrimidin-3ylamino)ethanol; 2-[(3-aminopyrazolo[1,5-a]pyrimidin7-yl)(2-hydroxyethyl)amino]ethanol; 2-[(7-aminopyrazolo[1,5-a]pyrimidin-3-yl)(2hydroxyethyl)amino]ethanol; 5,6-dimethylpyrazolo[1,5-a]pyrimidine-3,7-diamine; 2,6-dimethylpyrazolo[1,5-a]pyrimidine-3,7-diamine; 2,5,N7,N7-tetramethylpyrazolo[1,5-a]pyrimidine-3-amino-5-methyl7-(imidazolylpropylamino)pyrazolo[1,5-a]pyrimidine; and their addition salts and their tautomeric forms, when a tautomeric equilibrium exists, and their addition salts with an acid.

Mention may more particularly be made, among the pyrazole derivatives, of the compounds disclosed in patents DE 3 843 892 and DE 4 133 957 and patent applications WO 94/08969, WO 94/08970, FR-A-2 733 749 and DE 195 43 988, such as 4,5-diamino-1-methylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tertbutyl-3-methylpyrazole, 4,5-diamino-1-(β-hydroxyethyl)-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl3-(hydroxymethyl)pyrazole, 4,5-diamino-3-hydroxymethyl-1-methylpyrazole, 4,5-diamino-3-hydroxymethyl-1-isopropylpyrazole, 4,5-diamino-3-methyl1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-(methylamino)pyrazole, 3,5-diamino-4-(β-hydroxyethyl)amino-1-methylpyrazole, and their addition salts with an acid.

Generally, the oxidation bases preferably represent from 0.0005 to 12% by weight approximately of the total weight of the composition and more preferably still from 0.005 to 8% by weight approximately of this weight.

More preferably still, the keratinous fibers are dyed with at least one oxidation base and at least one coupler in the presence of an oxidizing agent.

Consequently, in the first compartment of the kit where it is recommended to use a conventional oxidation dye, there will be found at least one oxidation base and at least one coupler.

Mention may in particular be made, among these couplers, of meta-aminophenols, meta-phenylenediamines, meta-diphenols, heterocyclic couplers, such as, for example, indole derivatives, indoline derivatives, naphthalene derivatives, sesamol and its derivatives, pyridine derivatives, pyrazolotriazole derivatives, pyrazolones, and their addition salts with an acid.

These couplers are chosen more particularly from 2,4-diamino-1-(β-hydroxyethyloxy)benzene, 2-methyl-5-aminophenol, 5-N-(β-hydroxyethyl) amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethyl amino)-1-methoxybenzene, 1,3-diaminobenzene, 1,3-bis(2,4-diaminophenoxy) propane, sesamol, 1-amino-2-methoxy-4,5-methylenedioxybenzene, α-naphthol, 1-acetoxy-2-methylnaphthalene, 2-methyl-1-naphthol, 6-hydroxyindole, 4-hydroxyindole, 4-hydroxy-N-methylindole, 6-hydroxyindoline, 2,6-dihydroxy-4-methylpyridine, 1H-3-methylpyrazole-5-one, 1-phenyl-3-methylpyrazole-5-one, 2-amino-3-hydroxypyridine, 3,6-dimethylpyrazolo[3,2-c]-1,2,4-triazole, 2,6-dimethylpyrazolo[1,5-b]-1,2,4-triazole, and their addition salts with an acid.

Generally, the coupler or couplers preferably represent from 0.0001 to 15% by weight approximately of the total weight of the dyeing composition and more preferably still from 0.001 to 10% approximately.

Generally, the addition salts with an acid of the oxidation bases and of the couplers are chosen from hydrochlorides, hydrobromides and sulfates and tartrates, lactates and acetates.

The colorings obtained with conventional oxidation dyes can be given shades by the addition of direct dyes; thus, in addition, at least one direct dye can also be introduced into the first compartment of the kit or into another separate compartment.

In the case of a direct dyeing, the fibers are dyed beforehand with at least one direct dye.

Thus, in the first compartment of the kit where it is recommended to use direct dyeing, there will be found at least one direct dye.

The direct dyes which can be used to vary the shades obtained by means of a conventional oxidation dye or to dye fibers by a direct dyeing process can be chosen in particular from neutral, acidic or cationic nitrobenzene direct dyes, neutral, acidic or cationic azo direct dyes, neutral, acidic or cationic methine direct dyes, neutral, acidic or cationic quinone direct dyes and in particular anthraquinone direct dyes, azine direct dyes, triarylmethane direct dyes, indoamine direct dyes and natural direct dyes.

Mention may be made, among benzene direct dyes, without implied limitation, of the following compounds:
1,4-diamino-2-nitrobenzene
1-amino-2-nitro-4-(β-hydroxyethylamino)benzene
1-amino-2-nitro-4-bis(β-hydroxyethyl)aminobenzene
1,4-bis(β-hydroxyethylamino)-2-nitrobenzene
1-β-hydroxyethylamino-2-nitro-4-bis((β-hydroxyethylamino)benzene
1-β-hydroxyethylamino-2-nitro-4-aminobenzene
1-β-hydroxyethylamino-2-nitro-4-(ethyl)(β-hydroxyethyl)aminobenzene
1-amino-3-methyl-4-β-hydroxyethylamino-6-nitrobenzene
1-amino-2-nitro-4-β-hydroxyethylamino-5-chlorobenzene
1,2-diamino-4-nitrobenzene
1-amino-2-β-hydroxyethylamino-5-nitrobenzene
1,2-bis(β-hydroxyethylamino)-4-nitrobenzene
1-amino-2-[tris(hydroxymethyl)methylamino]-5-nitrobenzene
1-hydroxy-2-amino-5-nitrobenzene
1-hydroxy-2-amino-4-nitrobenzene
1-hydroxy-3-nitro-4-aminobenzene
1-hydroxy-2-amino-4,6-dinitrobenzene
1-β-hydroxyethyloxy-2-p-hydroxyethylamino-5-nitrobenzene
1-methoxy-2-β-hydroxyethylamino-5-nitrobenzene
1-β-hydroxyethyloxy-3-methylamino-4-nitrobenzene
1-β,γ-dihydroxypropyloxy-3-methylamino-4-nitrobenzene
1-β-hydroxyethylamino-4-β,γ-dihydroxypropyloxy-2-nitrobenzene
1-β,γ-dihydroxypropylamino-4-trifluoromethyl-2-nitrobenzene
1-β-hydroxyethylamino-4-trifluoromethyl-2-nitrobenzene
1-β-hydroxyethylamino-3-methyl-2-nitrobenzene
1-β-aminoethylamino-5-methoxy-2-nitrobenzene
1-hydroxy-2-chloro-6-ethylamino-4-nitrobenzene
1-hydroxy-2-chloro-6-amino-4-nitrobenzene
1-hydroxy-6-[bis(β-hydroxyethyl)amino]-3-nitrobenzene
1-β-hydroxyethylamino-2-nitrobenzene
1-hydroxy-4-β-hydroxyethylamino-3-nitrobenzene.

Mention may be made, among the azo direct dyes, of the cationic azo dyes disclosed in patent applications WO 95/15144, WO 95/01772 and EP-714 954, the contents of which form an integral part of the invention.

Mention may also be made, among the azo direct dyes, of the following dyes, described in the Colour Index International, 3rd edition:
Disperse Red 17; Acid Yellow 9; Acid Black 1; Basic Red 22; Basic Red 76; Basic Yellow 57; Basic Brown 16; Acid Yellow 36; Acid Orange 7; Acid Red 33; Acid Red 35; Basic Brown 17; Acid Yellow 23; Acid Orange 24; Disperse Black 9.

Mention may also be made of 1-(4′-aminodiphenylazo)-2-methyl-4-(bis(β-hydroxyethyl)amino]benzene and 4-hydroxy-3-(2-methoxyphenylazo)-1-naphthalenesulfonic acid.

Mention may more particularly be made, among the methine direct dyes, of cationic methine dyes, such as Basic Red 14, Basic Yellow 13 and Basic Yellow 29.

Mention may be made, among the quinone direct dyes, of the following dyes:
Disperse Red 15; Solvent Violet 13; Acid Violet 43; Disperse Violet 1; Disperse Violet 4; Disperse Blue 1; Disperse Violet 8; Disperse Blue 3; Disperse Red 11; Acid Blue 62; Disperse Blue 7; Basic Blue 22; Disperse Violet 15; Basic Blue 99, and the following compounds:
   1-N-methylmorpholiniumpropylamino-4-hydroxyanthraquinone
   1-aminopropylamino-4-(methylamino)anthraquinone
   1-aminopropylaminoanthraquinone
   5-β-hydroxyethyl-1,4-diaminoanthraquinone
   2-aminoethylaminoanthraquinone
   1,4-bis(β,γ-dihydroxypropylamino)anthraquinone.

Mention may be made, among azine direct dyes, of the following compounds, such as Basic Blue 17 and Basic Red 2.

Mention may be made, among the triarylmethane direct dyes, of the following compounds:
Basic Green 1; Acid Blue 9; Basic Violet 3; Basic Violet 14; Basic Blue 7; Acid Violet 49; Basic Blue 26; Acid Blue 7.

Mention may be made, among the indoamine direct dyes, of the following compounds:
2-β-hydroxyethylamino-5-[bis(-4′-hydroxyethyl) amino]anilino-1,4-benzoquinone;
2-β-hydroxyethylamino-5-(2′-methoxy-4′-amino)anilino-1,4-benzoquinone;
3-N(2′-chloro-4′-hydroxy)-phenyl-acetylamino-6-methoxy-1,4-benzoquinone imine
3-N(3′-chloro-4′methylamino)-phenyl-ureido-6-methyl-1,4-benzoquinone imine;
3-[4′-N-(ethyl, carbamoylmethyl)amino]-phenyl-ureido-6-methyl-1,4-benzoquinone imine.

Mention may be made, among the natural direct dyes, of lawsone, juglone, alizarin, purpurin, carminic acid, kermesic acid, purpurogallin, protocatechaldehyde, indigo, isatin, curcumin, spinulosin or apigenidin. Use may also be made of the extracts or decoctions comprising these natural dyes and in particular cataplasms or henna-based extracts.

In the case of a lightening dyeing, the fibers are dyed beforehand with at least one direct dye in the presence of an oxidizing agent.

Thus, in the first compartment of the kit where it is recommended to use lightening dyeing, there will be found at least one direct dye.

Mention may be made, among the direct dyes which it is preferable to use in lightening dyeing, of the cationic methine dyes described above and more particularly still cationic dyes, such as those disclosed in the European patent application of the Applicant Company No. 1025834, which, in an oxidizing medium, generate highly chromatic shades.

Said cationic direct dyes preferably used in lightening direct dyeing are chosen in particular from those of following formula (I) and those of following formulae (II)a, (II)b, (III)a, (III)b and (IV) to (VII) and their mesomeric forms:
(i) dyes of formulae (I), (II)a, (II)b, (III)a and (III)b: 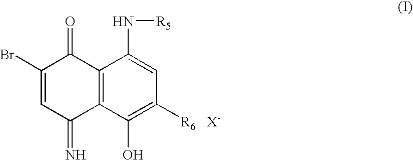 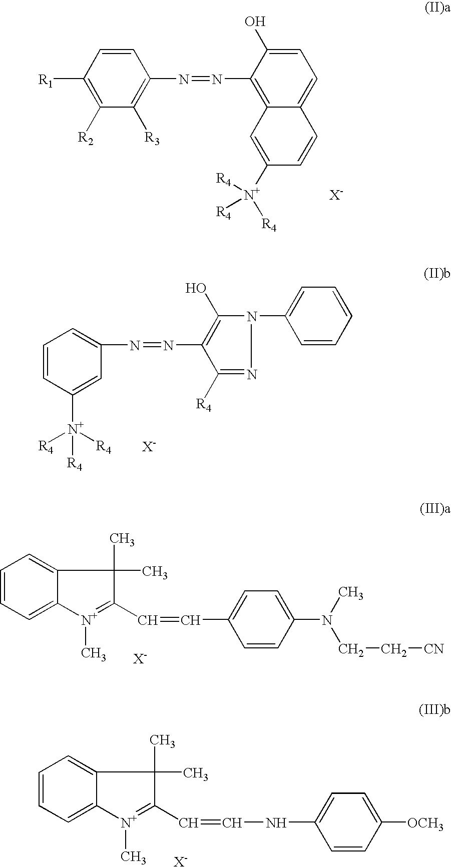
   in which formulae (I), (II)a, (II)b, (III)a and (III)b:
R₁ represents a hydrogen atom or an amino radical;
R₂ represents a hydrogen atom or a nitro group;
R₃ represents a hydrogen atom, a nitro group or a C₁-C₄ alkoxy radical;
R₄ represents a C₁-C₄ alkyl radical;
R₅ represents a hydrogen atom or a para-triC₁-C₄alkylammoniophenyl group;
R₆ represents a bromine atom or an NH-para-triC₁-C₄alkylammoniophenyl group;
X⁻ represents an anion preferably chosen from chloride, methyl sulfate and acetate;
(ii) dyes of formulae (IV), (V), (VI), (VI′) and (VII):
   a) the compounds of following formula (IV): 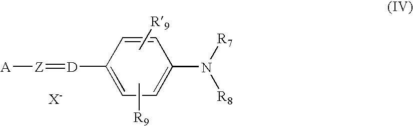
      in which:
Z and D represent, which are identical or different, a nitrogen atom or the —CH— radical,
R₇ and R₈, which are identical or different, represent a hydrogen atom; a C₁-C₄ alkyl radical which can be substituted by a —CN, —OH or —NH₂ radical or form, with a carbon atom of the benzene ring, an optionally oxygen-comprising or nitrogen-comprising heterocycle which can be substituted by one or more C₁-C₄ alkyl radicals; or a 4′-aminophenyl radical,
R₉ and R′₉ , which are identical or different, represent a hydrogen atom, a halogen atom chosen from chlorine, bromine, iodine and fluorine, a cyano radical, a C₁-C₄ alkyl radical, a C₁-C₄ alkoxy radical or an acetyloxy radical,
X⁻ represents an anion preferably chosen from chloride, methyl sulfate and acetate,
A represents a group chosen by the following structures A1 to A19: 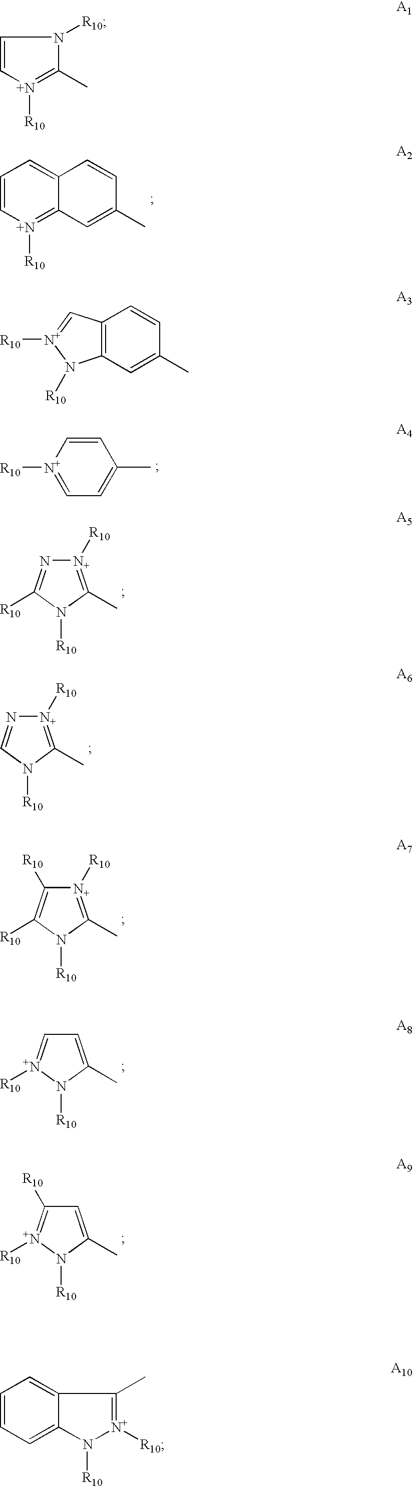 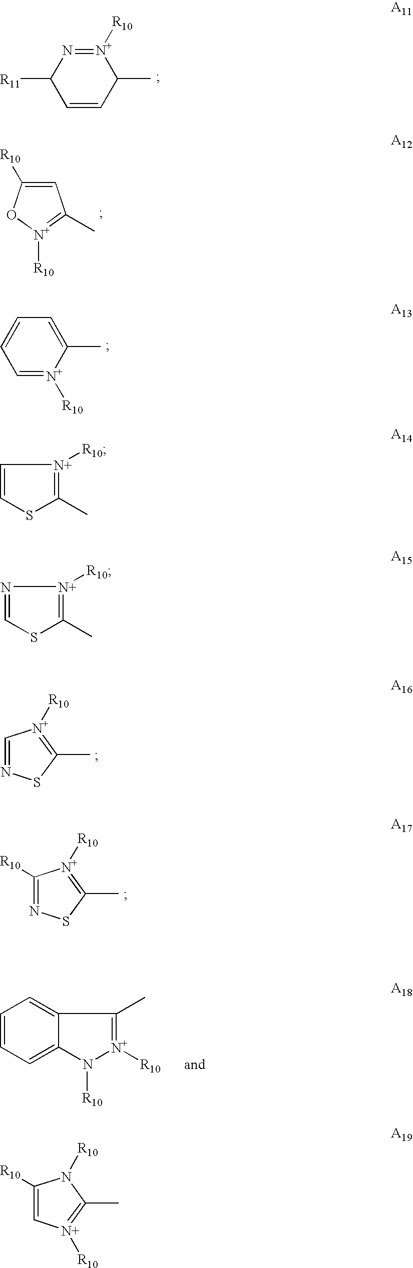
   in which R₁₀ represents a C₁-C₄ alkyl radical which can be substituted by a hydroxyl radical and R₁₁ represents a C₁-C₄ alkoxy radical;
   b) the compounds of following formula (V): 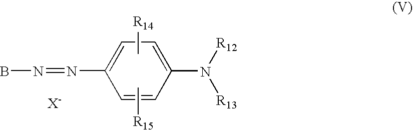
      in which:
R₁₂ represents a hydrogen or a C₁-C₄ alkyl radical,
R₁₃ represents a hydrogen atom, an alkyl radical which can be substituted by a —CN radical or by an amino group, or a 4′-aminophenyl radical or forms, with R₁₂, an optionally oxygen-comprising and/or nitrogen-comprising heterocycle which can be substituted by a C₁-C₄ alkyl radical,
R₁₄ and R₁₅, which are identical or different, represent a hydrogen atom, a halogen atom, such as bromine, chlorine, iodine or fluorine, a C₁-C₄ alkyl radical, a C₁-C₄ alkoxy radical or a -CN radical,
X⁻ represents an anion preferably chosen from chloride, methyl sulfate and aetate.
B represents a group chosen by the following structures B1 to B6: 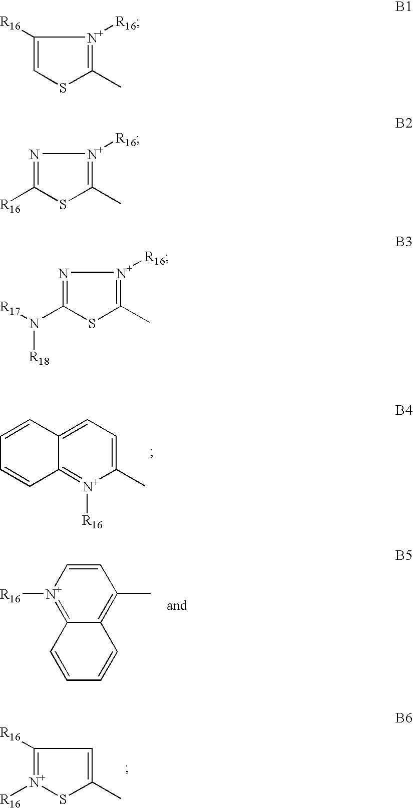
   in which R₁₆ represents a C₁-C₄ alkyl radical and R₁₇ and R₁₈, which are identical or different, represent a hydrogen atom or a C₁-C₄ alkyl radical;
   c) the compounds of following formulae (VI) and (VI′): 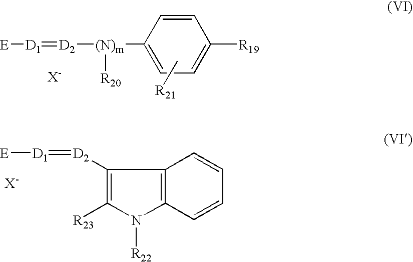
      in which:
R₁₉ represents a hydrogen atom, a C₁-C₄ alkoxy radical, a halogen atom, such as bromine, chlorine, iodine or fluorine, or an amino radical,
R₂₀ represents a hydrogen atom or a C₁-C₄ alkyl radical or forms, with a carbon atom of the benzene ring, a heterocycle which optionally comprises oxygen and/or is substituted by one or more C₁-C₄ alkyl groups,
R₂₁ represents a hydrogen atom or a halogen atom, such as bromine, chlorine, iodine or fluorine,
R₂₂ and R₂₃, which are identical or different, represent a hydrogen atom or a C₁-C₄ alkyl radical,
D₁ and D₂, which are identical or different, represent a nitrogen atom or the —CH group,
m=0 or 1,
X⁻ represents an anion preferably chosen from chloride, methyl sulfate and acetate,
E represents a group chosen by the following structures E1 to E8: 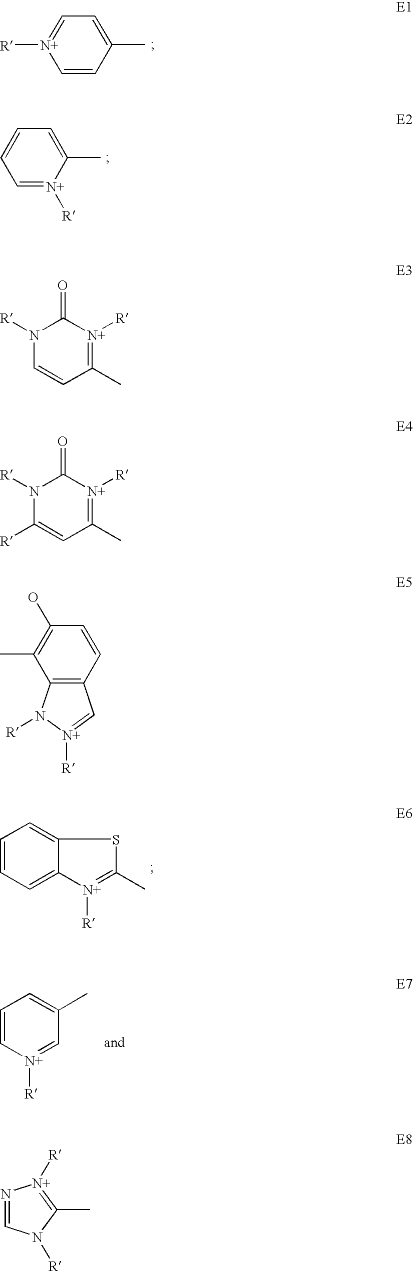
   in which R′ represents a C₁-C₄ alkyl radical;
when m=0 and when D₁₃ represents a nitrogen atom, then E can also denote a group with the following structure E9: 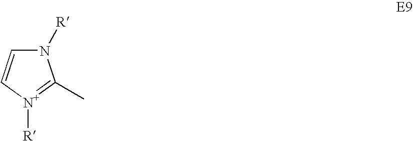
   in which R′ represents a C₁-C₄ alkyl radical.
   d) the compounds of following formula (VII):
      G-N═N-J (VII)
      in which:
th symbol G represents a group chosen from the following structures G₁ to G₃: 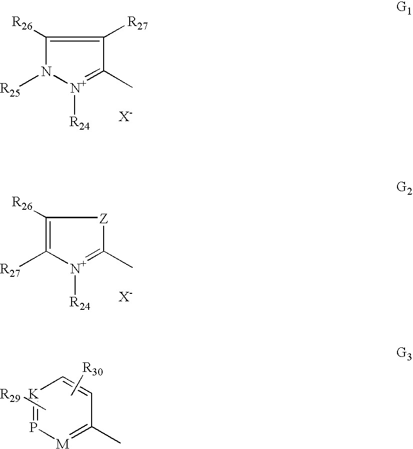
   in which structures G₁ to G₃,
R₂₄ denotes a C₁-C₄ alkyl radical or a phenyl radical which can be substituted by a C₁-C₄ alkyl radical or a halogen atom chosen from chlorine, bromine, iodine and fluorine;
R₂₅ denotes a C₁-C₄ alkyl radical or a phenyl radical;
R₂₆ and R₂₇, which are identical or different, represent a C₁-C₄ alkyl radical or a phenyl radical or together form, in G₁, a benzene ring substituted by one or more C₁-C₄ alkyl, C₁-C₄ alkoxy or NO₂ radicals or together form, in G₂, a benzene ring optionally substituted by one or more C₁-C₄ alkyl, C₁-C₄ alkoxy or NO₂ radicals;
R₂₆ can additionally denote a hydrogen atom;
Z denotes an oxygen or sulfur atom or an —NR₂₅ group;
M represents a —CH, —CR (R denoting C₁-C₄ alkyl) or —NR₂₈(X⁻)ᵣ group;
K represents a —CH, —CR (R denoting C₁-C₄ alkyl) or —NR₂₈(X⁻)ᵣ group;
P represents a —CH, —CR (R denoting C₁-C₄ alkyl) or —NR₂₈(X⁻)ᵣ group; r denotes zero or 1;
R₂₈ represents an O⁻ atom, a C₁-C₄ alkoxy radical or a C₁-C₄ alkyl radical;
R₂₉ and R₃₀, which are identical or different, represent a hydrogen atom, a halogen atom chosen from chlorine, bromine, iodine and fluorine, a C₁-C₄ alkyl radical, a C₁-C₄ alkoxy radical or an —NO₂ radical;
X⁻ represents an anion preferably chosen from chloride, iodide, methyl sulfate, ethyl sulfate, acetate and perchlorate;
the symbol J represents:
   (a) a group with the following structure J₁: 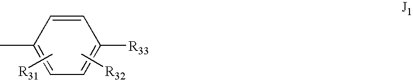
      in which structure J₁,
R₃₁, represents a hydrogen atom, a halogen atom chosen from chlorine, bromine, iodine and fluorine, a C₁-C₄ alkyl radical, a C₁-C₄ alkoxy radical or an —OH, —NO₂, —NHR₃₄, —NR₃₅R₃₆ or —NHCO(C₁-C₄)alkyl radical or forms, with R₃₂, a 5- or 6-membered ring which may or may not comprise one or more heteroatoms chosen from nitrogen, oxygen or sulfur;
R₃₂ represents a hydrogen atom, a halogen atom chosen from chlorine, bromine, iodine and fluorine, a C₁-C₄ alkyl radical or a C₁-C₄ alkoxy radical,
or forms, with R₃₃ or R₃₄, a 5- or 6-membered ring which may or may not comprise one or more heteroatoms chosen from nitrogen, oxygen or sulfur;
R₃₃ represents a hydrogen atom, an —OH radical, an —NHR₃₄ radical or an —NR₃₅R₃₆ radical;
R₃₄ represents a hydrogen atom, a C₁-C₄ alkyl radical, a C₁-C₄ monohydroxyalkyl radical, a C₂-C₄ polyhydroxyalkyl radical or a phenyl radical;
R₃₅ and R₃₆, which are identical or different, represent a C₁-C₄ alkyl radical, a C₁-C₄ monohydroxyalkyl radical or a C₂-C₄ polyhydroxyalkyl radical;
   (b) a 5- or 6-membered nitrogenous heterocyclic group which can include other heteroatoms and/or carbonyl groups and which can be substituted by one or more C₁-C₄alkyl, amino or phenyl radicals, and in particular a group with the following structure J₂: 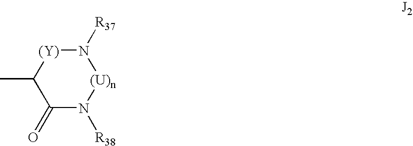
      in which structure J₂,
R₃₇ and R₃₈, which are identical or different, represent a hydrogen atom, a C₁₃-C₁₀ alkyl radical or a phenyl radical;
Y denotes the —CO— radical or the 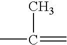
   radical;
n=0 or 1, with, when n denotes 1, U denotes the —CO— radical.

Mention may more particularly be made, among the cationic direct dyes of formula (IV), of the compounds corresponding to the following structures (IV1) to

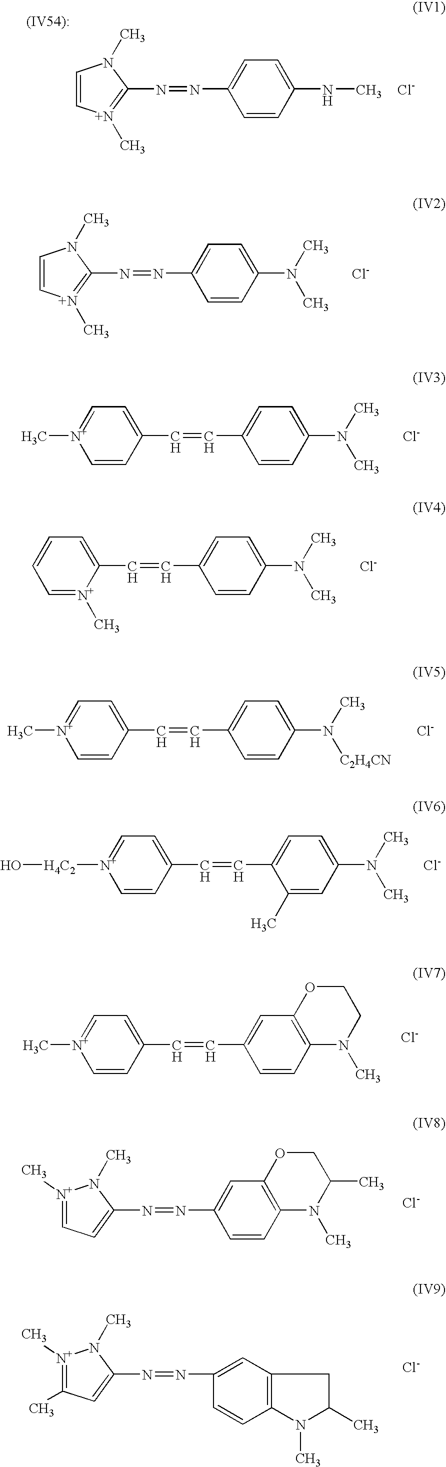

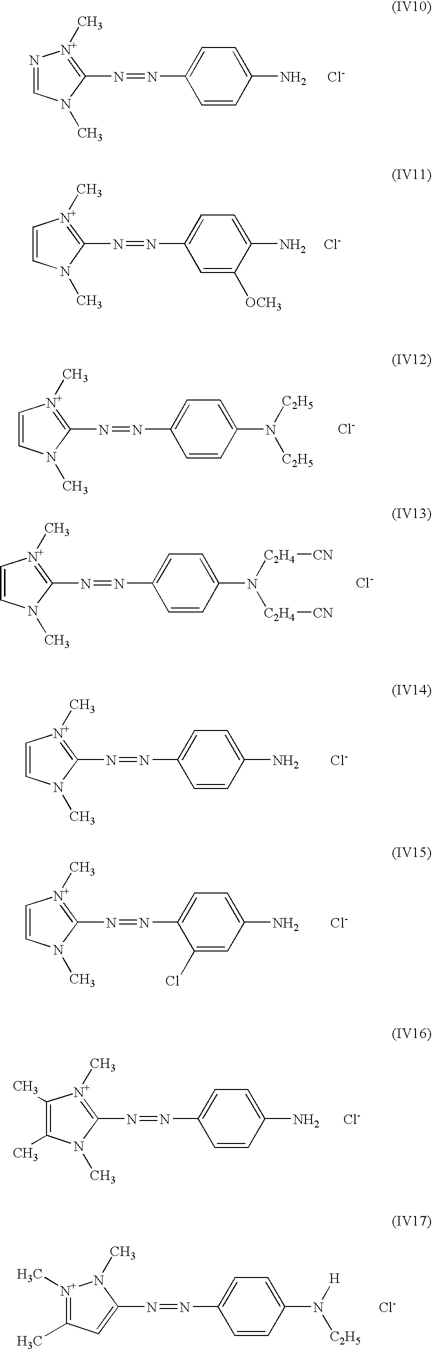

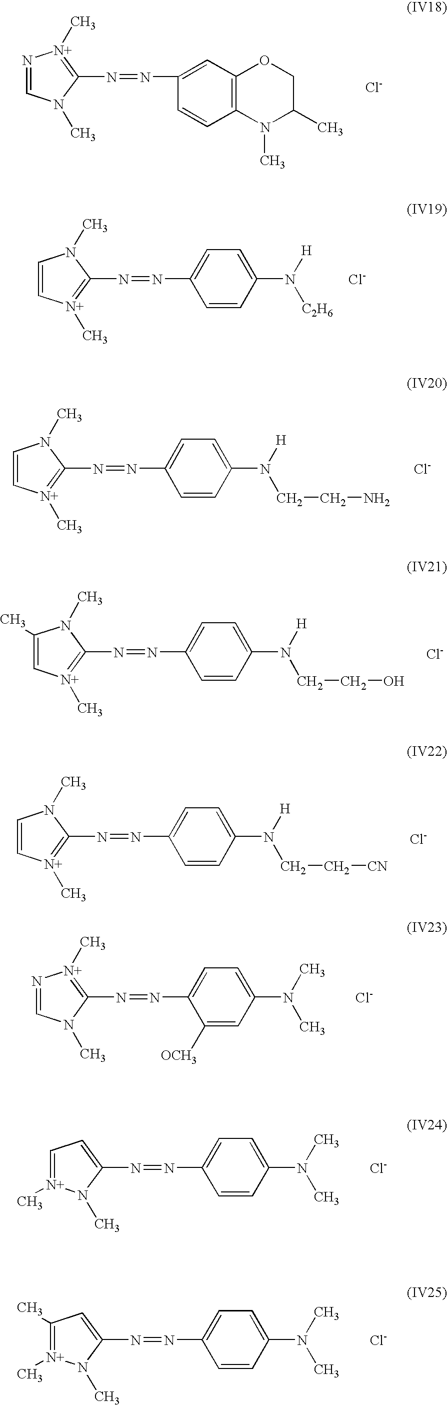

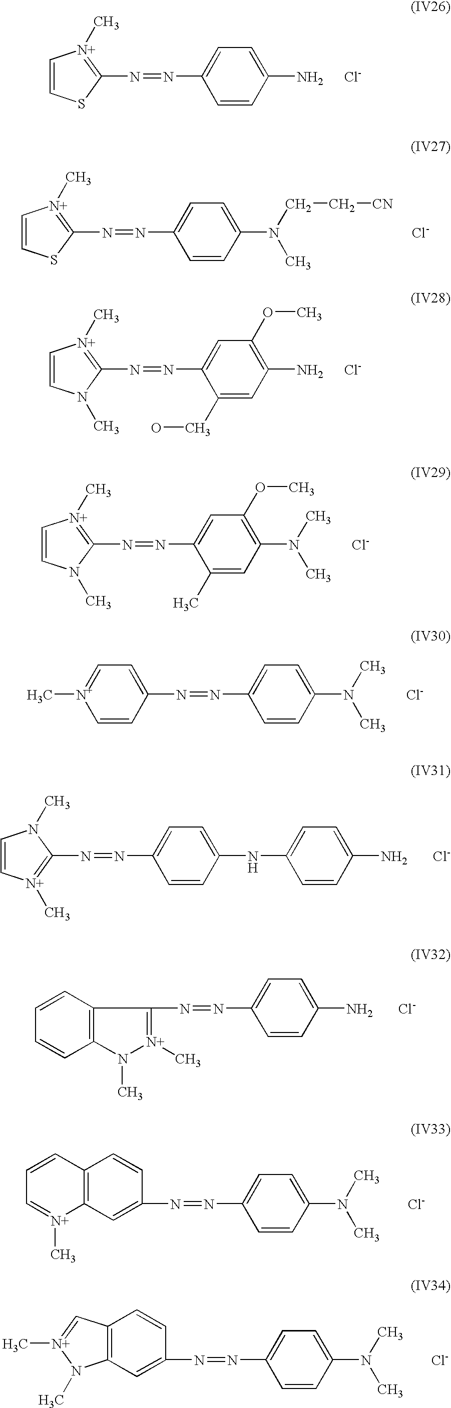

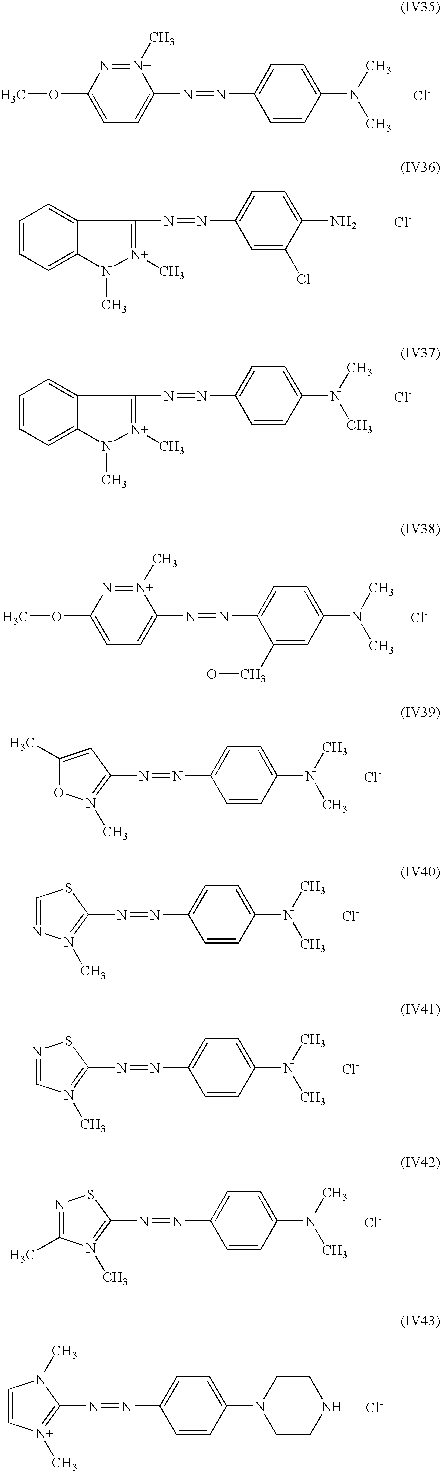

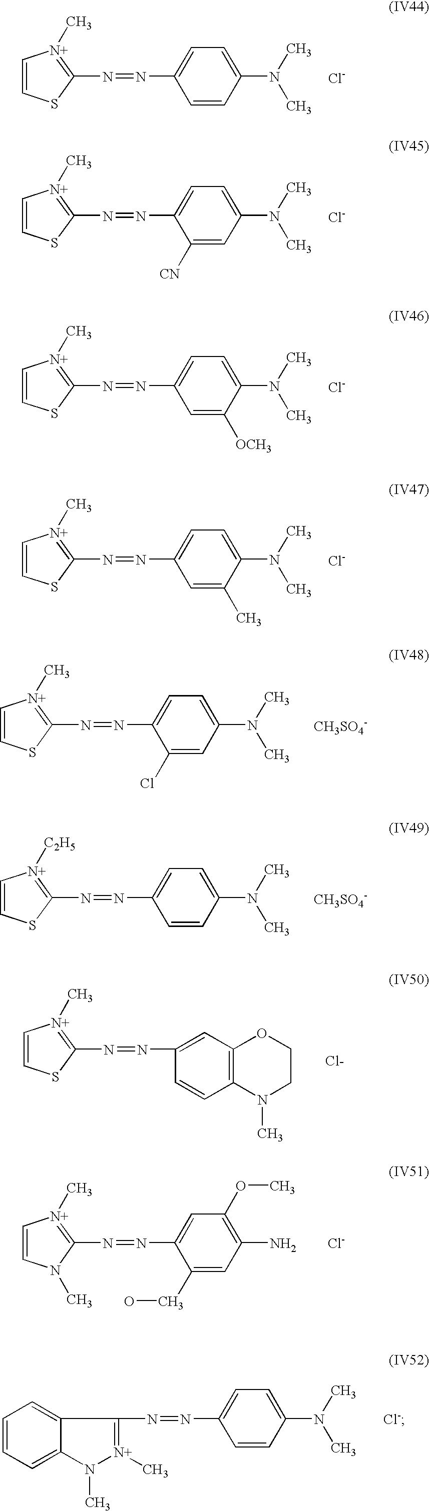

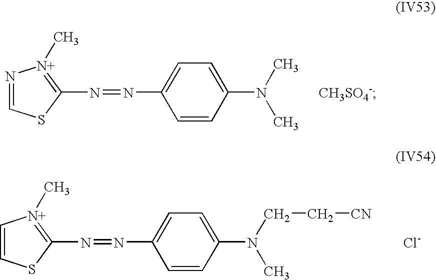

Preference is very particularly given, among the compounds with the structures (IV1) to (IV54) described above, to the compounds corresponding to the structures (IV1), (IV2), (IV14) and (IV31).

Mention may more particularly be made, among the cationic direct dyes of formula (V), to the compounds corresponding to the following structures (V1) to (V9):

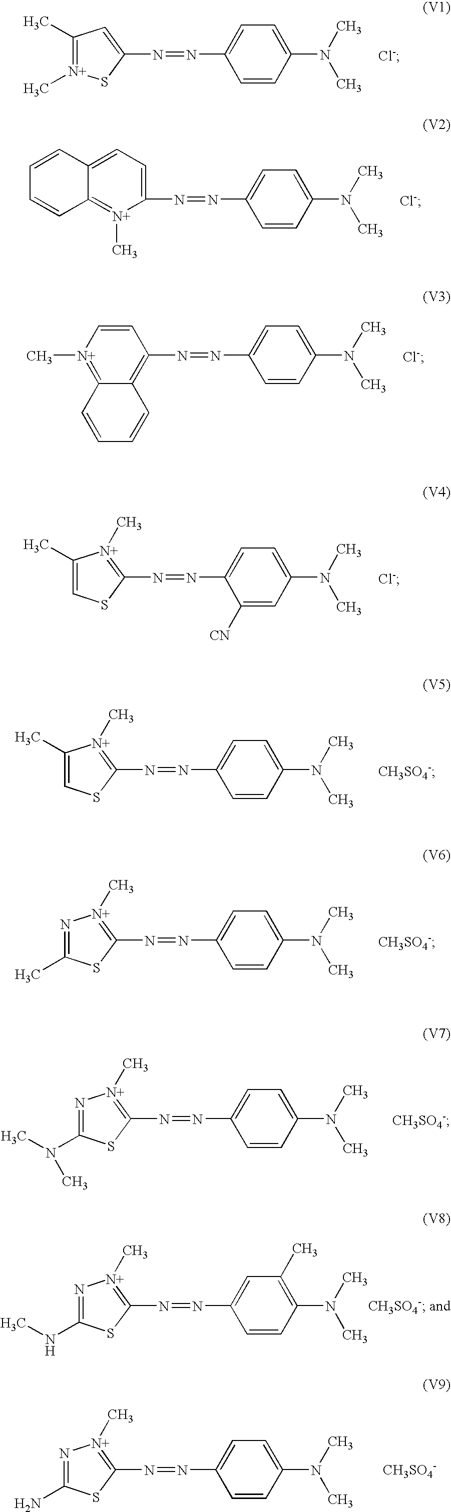

Mention may more particularly be made, among the cationic direct dyes of formula (VI) which can be used in the dyeing compositions in accordance with the invention, of the compounds corresponding to the following structures (VI1) to (VI18):

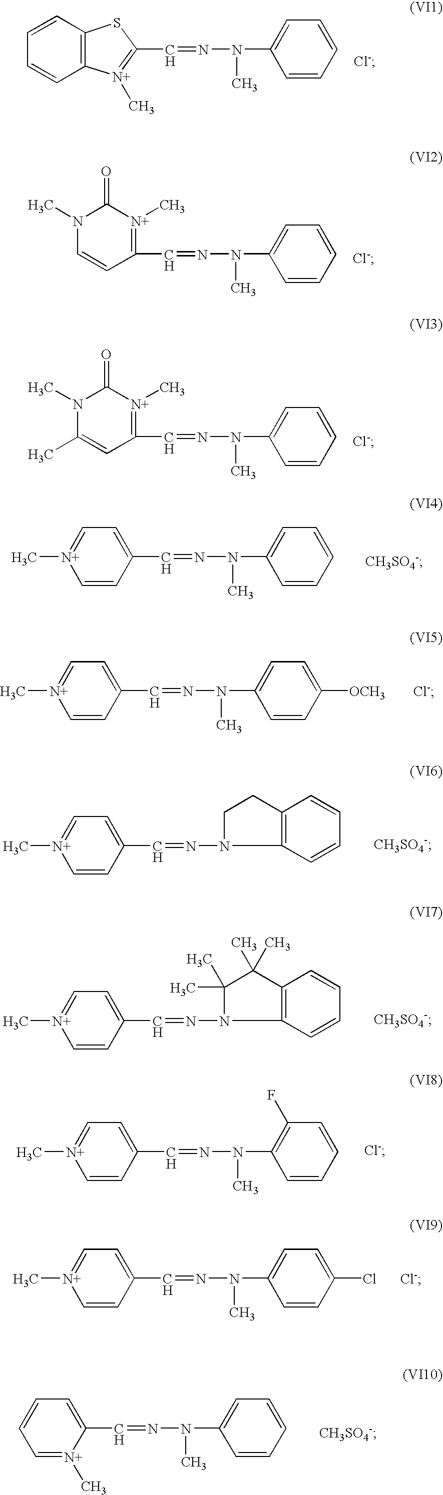

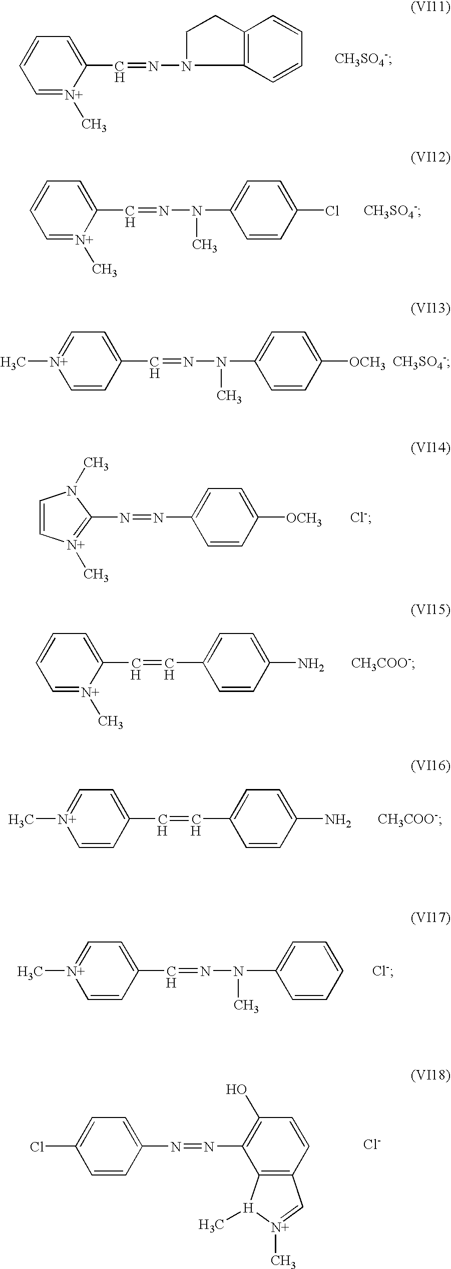

Preference is very particularly given, among the specific compounds with the structures (VI1) to (VI18) described above, to the compounds corresponding to the structues (VI4), (VI5) and (VI13).

Mention may more particularly be made, among the cationic direct dyes of formula (VI′), of the compounds corresponding to the following structures (VI′1) to (VI′3):

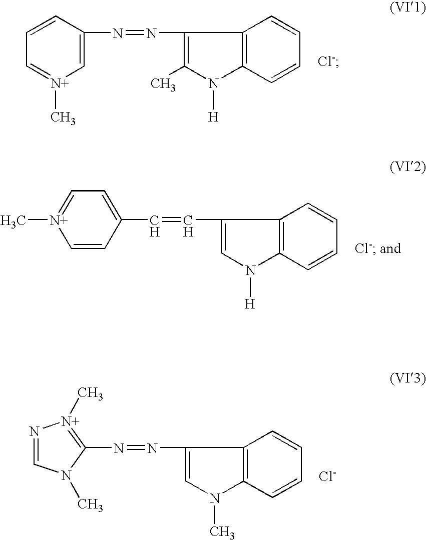

Mention may more particularly be made, among the cationic direct dyes of formula (VII), of the compounds with the following structures (VII₁) to (VII₇₇):

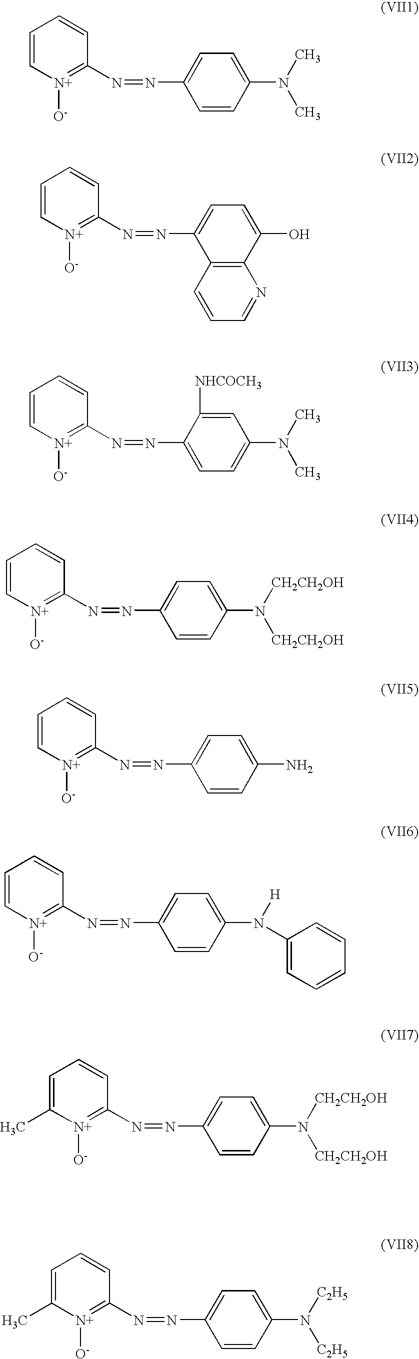

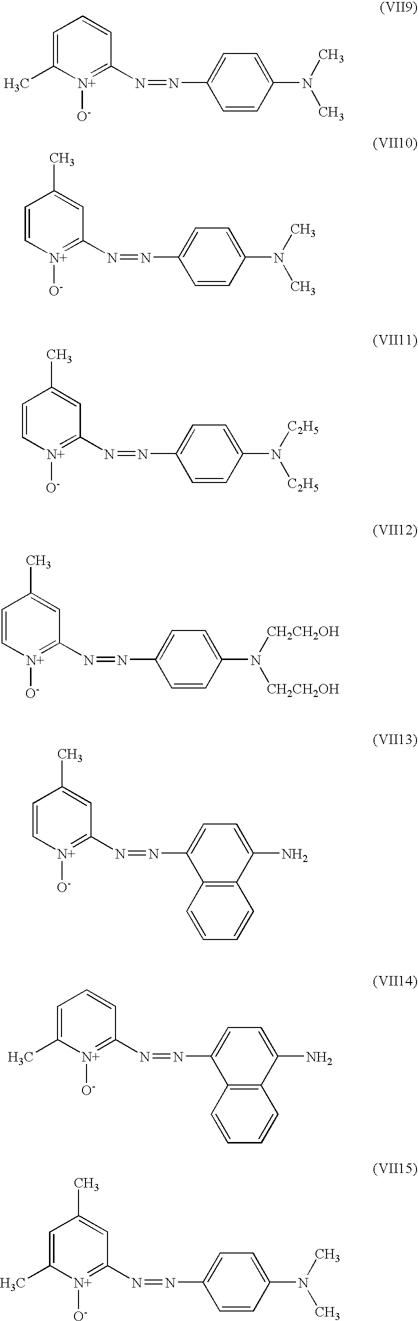

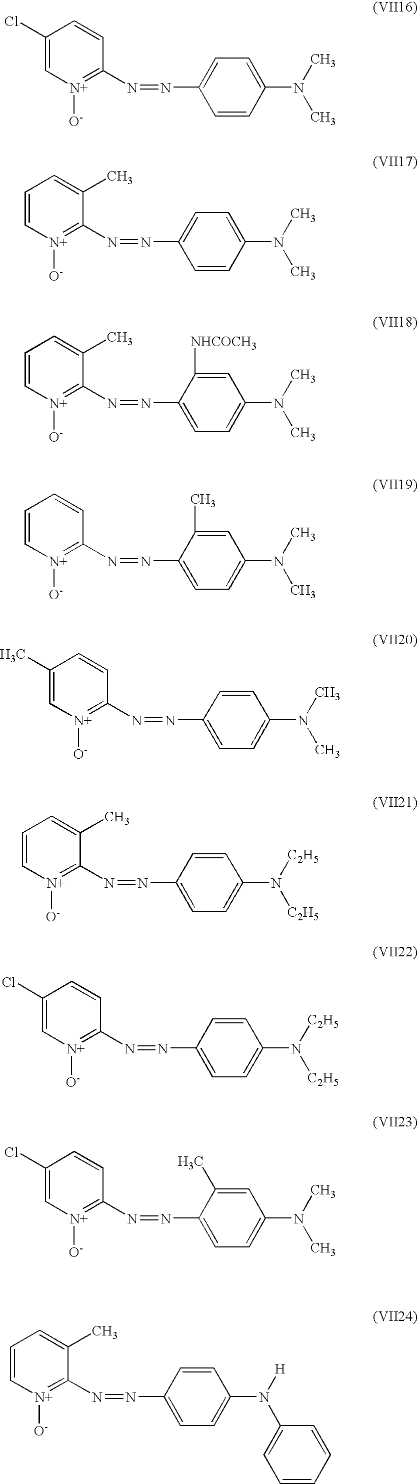

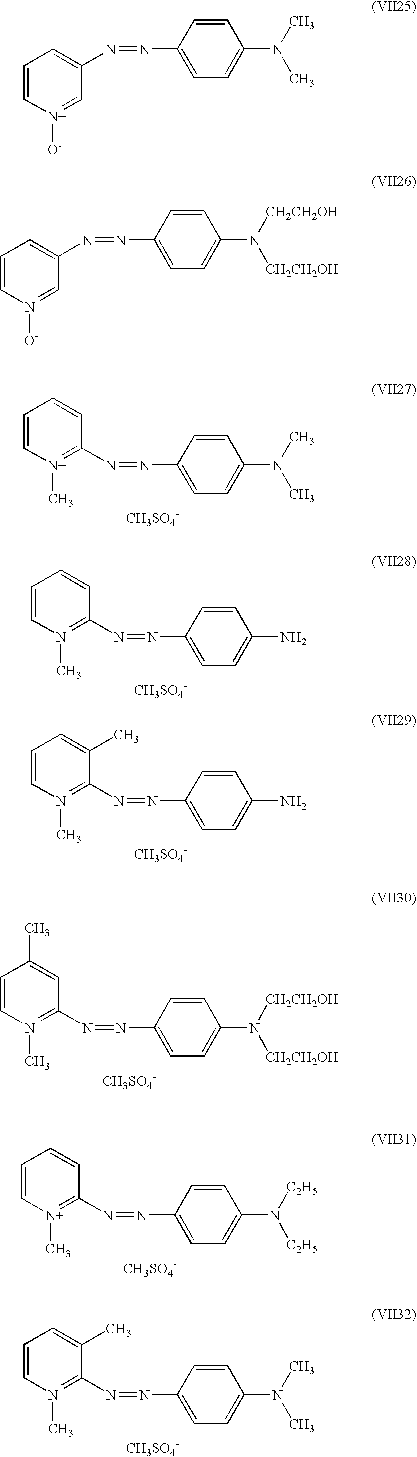

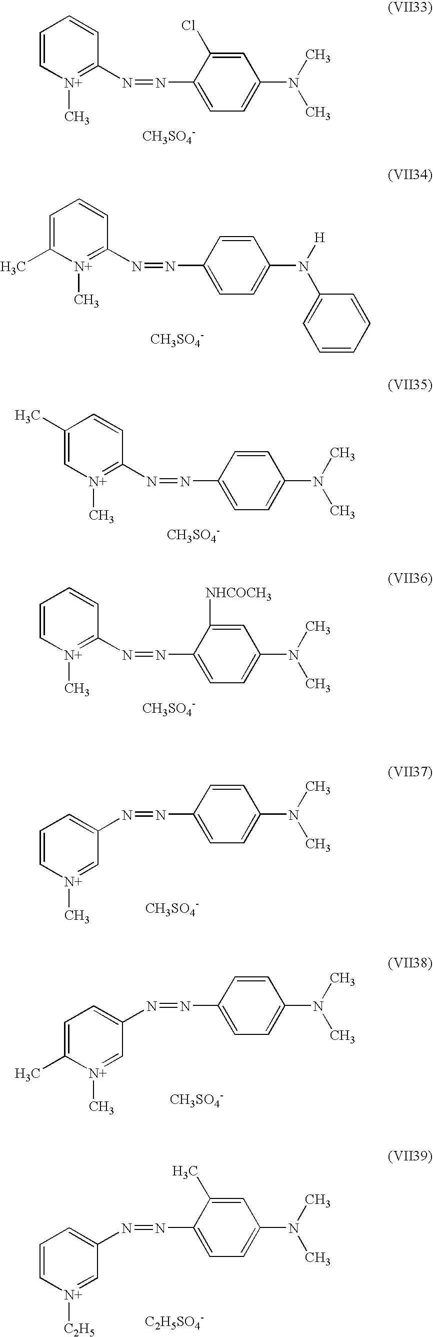

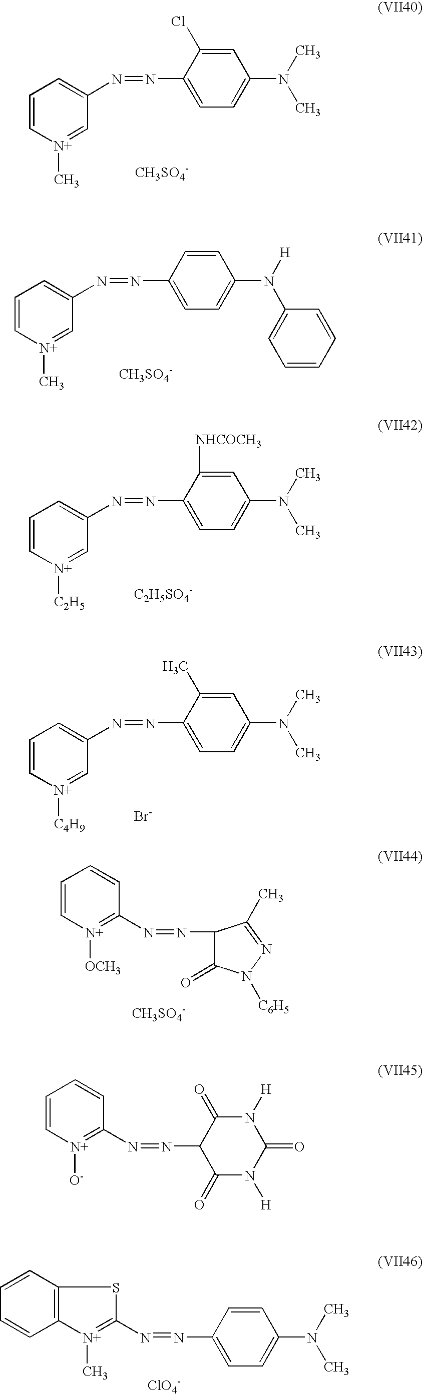

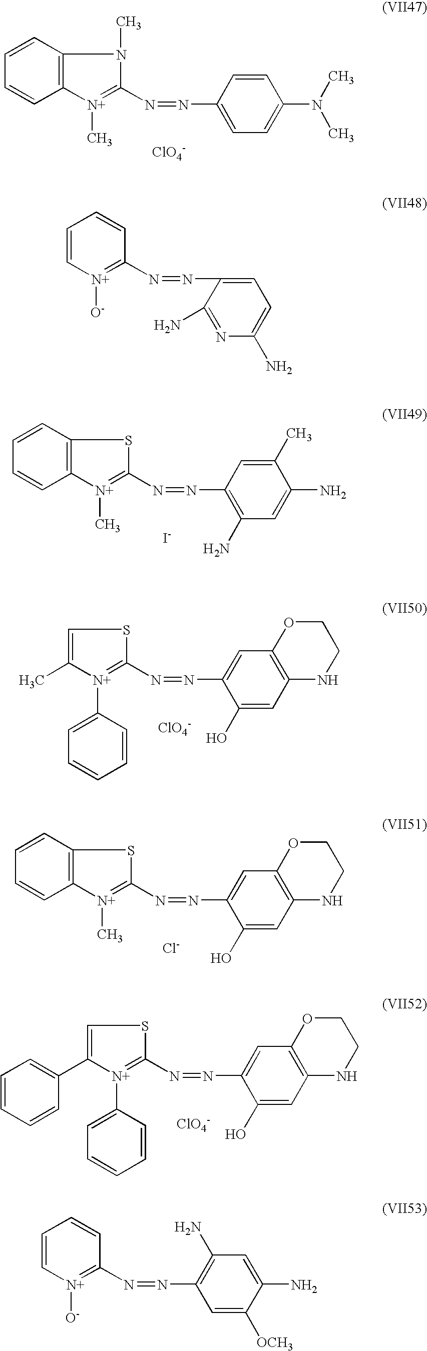

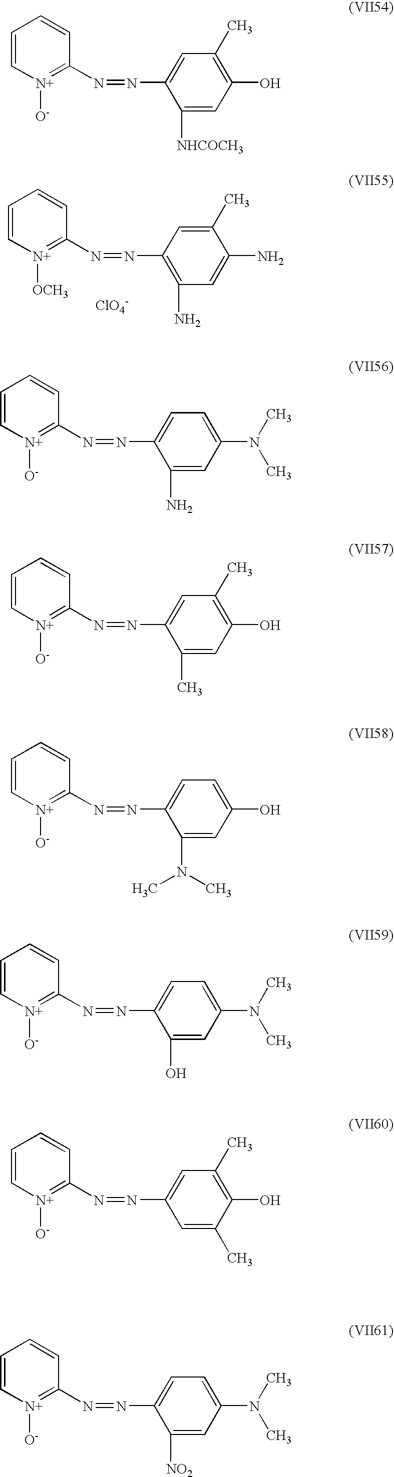

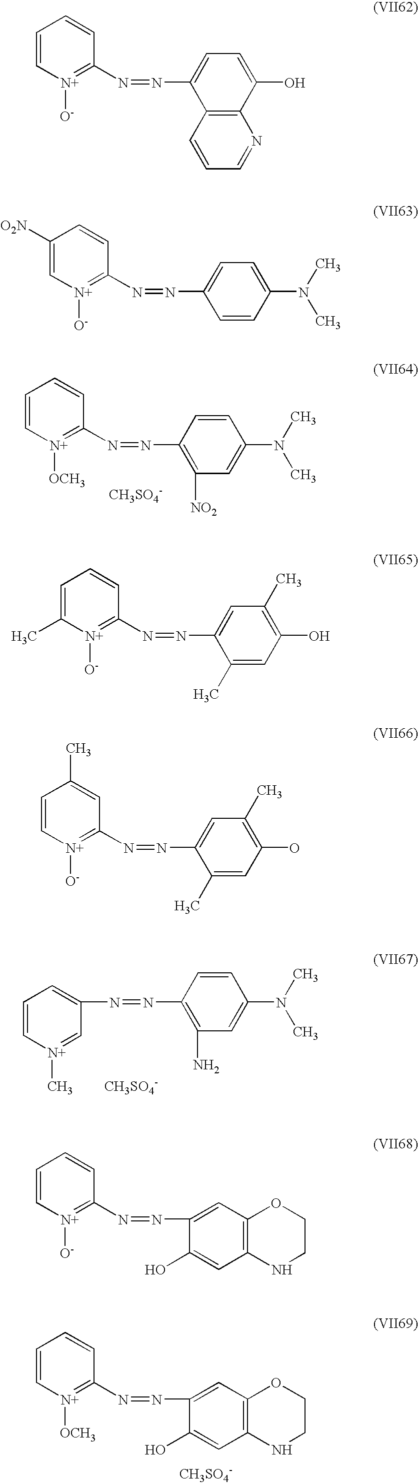

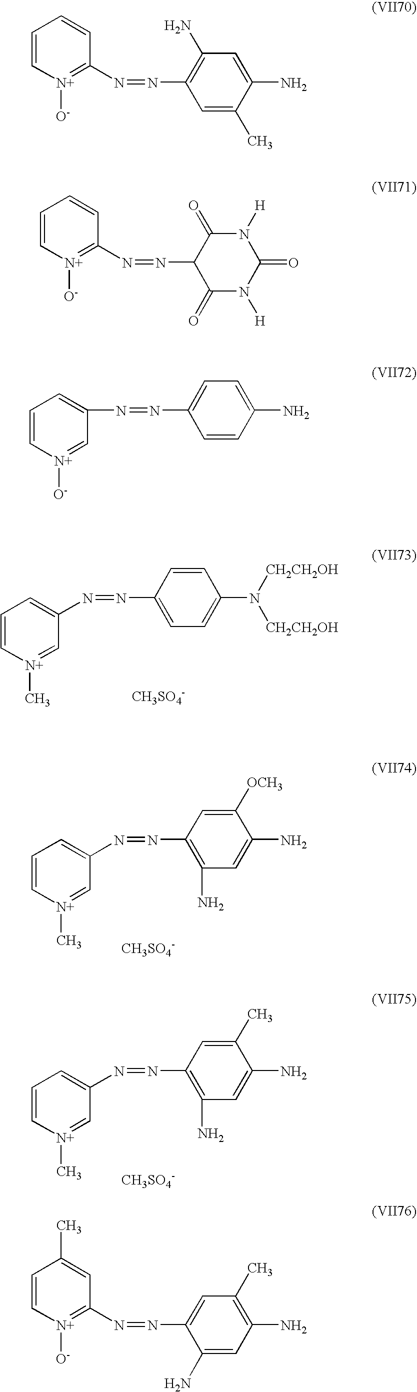

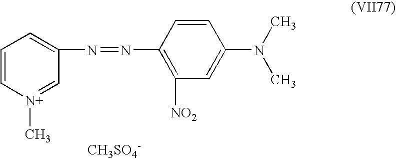

All these particularly persistent dyes of formulae (I) to (VII) can be stripped by means of the bleaching composition according to the present invention and very particularly the dyes of formulae (IV2) (=Basic Red 51), (IV14) (=Basic Orange 31) and (VI4) (=Basic Yellow 87).

The direct dye or dyes preferably represent from 0.001 to 20% by weight approximately of the total weight of the composition for oxidizing or nonoxidizing dyeing and more preferably still from 0.005 to 10% by weight approximately.

In the multicompartment kit, the oxidizing agent necessary for carrying out the oxidation dyeing or the lightening dyeing is separated from the oxidation dye or dyes or from the cationic direct dye or dyes. It is preferably chosen from hydrogen peroxide, urea hydrogen peroxide, alkali metal bromates or ferricyanides, or persalts, such as perborates and persulfates, the use of hydrogen peroxide being particularly preferred. This oxidizing agent is advantageously composed of an aqueous hydrogen peroxide solution, the assay of which can vary more particularly from approximately 1 to 40 volumes and more preferably still from approximately 5 to 40.

Use may also be made, as oxidizing agent, of one or more oxidation/reduction enzymes, such as 4-electron oxidoreductases (such as laccases), peroxidases and 2-electron oxidoreductases (such as uricase), if appropriate in the presence of their respective donor or cofactor.

The medium appropriate for bleaching (or support) of the bleaching composition in accordance with the invention is generally composed of water or of a mixture of water and of at least one organic solvent in order to dissolve the compounds which would be insufficiently soluble in water. Mention may be made, for example, as organic solvent, of C₁-C₄ alkanols, such as ethanol and isopropanol; glycerol; glycols and glycol ethers, such as 2-butoxyethanol, propylene glycol, dipropylene glycol, hexylene glycol, propylene glycol monomethyl ether, diethylene glycol monoethyl ether and diethylene glycol monomethyl ether; and aromatic alcohols, such as benzyl alcohol or phenoxyethanol, the analogous products and their mixtures.

Said solvents can then be present in proportions preferably of between 0.5 to 20% and more particularly from 2 to 10% by weight with respect to the total weight of the bleaching composition.

The pH of the bleaching composition in accordance with the invention is acidic and preferably between 1.8 and 6.

It is adjusted by acidifying or basifying agents in amounts ranging from 0.01 to 30% by weight of the total weight of the composition.

Mention may be made, among acidifying agents, by way of example, of inorganic or organic acids, such as etidronic acid, hydrochloric acid, orthophosphoric acid, sulfuric acid, carboxylic acids, such as acetic acid, tartaric acid, citric acid or lactic acid, and sulfonic acids.

Mention may be made, among basifying agents, by way of example, of ammonia, alkaline carbonates, alkanolamines, such as mono-, di- and triethanolamines, 2-methyl-2-amino-1-propanol and their derivatives, sodium hydroxide, potassium hydroxide and the compounds of following formula (K): 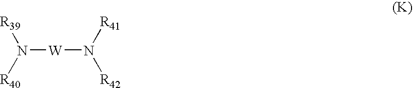
in which W is a propylene residue optionally substituted by a hydroxyl group or a C₁-C₄ alkyl radical; and R₃₉, R₄₀, P₄₁ and R₄₂, which are identical or different, represent a hydrogen atom, a C₁-C₄ alkyl radical or a C₁-C₄ hydroxyalkyl radical.

The bleaching composition in accordance with the invention can include at least one thickening agent in a proportion of between 0.01 and 10% and in particular between 0.1 and 5% approximately by weight of the total weight of the composition.

They are preferably chosen from cellulose derivatives, guar derivatives, gums of microbial or plant origin and synthetic thickeners.

Thickening Agents:

Mention may be made, among cellulose derivatives, of hydroxy(C₁-C₆)alkylcelluloses and carboxy(C₁-C₆)alkylcelluloses.

The hydroxy(C₁-C₆)alkylcelluloses are more particularly hydroxyethylcelluloses, such as those sold under the names Cellosize QP3L, Cellosize QP4400H, Cellosize QP30000H, Cellosize HEC30000A or Cellosize Polymer PCG10 by Amerchol or Natrosol 250HHR, Natrosol 250MR, Natrosol 250M, Natrosol 250HHXR, Natrosol 250HHX, Natrosol 250HR or Natrosol HX by Hercules or Tylose H1000 by Hoechst.

The hydroxy(C₁-C₆)alkylcelluloses are also more particularly hydroxypropylcelluloses, such as the products sold under the names Klucel E F, Klucel H, Klucel L H F, Klucel M F or Klucel G by Aqualon.

Use is preferably made, among the carboxy-(C₁-C₆)alkylcelluloses, of carboxymethylcellulose, of which may be mentioned the products sold under the names Blanose 7M8/SF, Blanose Raffinée 7M, Blanose 7LF, Blanose 7MF, Blanose 9M31F, Blanose 12M31XP, Blanose 12M31P, Blanose 9M31XF, Blanose 7H, Blanose 7M31 or Blanose 7H3SXF by Aqualon or Aquasorb A500 and Ambergum 1221 by Hercules or Cellogen HP810A and Cellogen HP6HS9 by Montello or Primellose by Avebe.

Mention may be made, among the guar derivatives, of unmodified or modified nonionic guar gums. The unmodified guar gums are, for example, the products sold under the name Vidogum GH 175 by Unipectine and under the names Meypro-Guar 50 and Jaguar C by Meyhall. The modified nonionic guar gums are modified in particular with C₁-C₆ hydroxyalkyl groups. Mention may be made, among hydroxyalkyl groups, by way of example, of the hydroxymethyl, hydroxyethyl, hydroxypropyl and hydroxybutyl groups.

Such nonionic guar gums optionally modified with hydroxyalkyl groups are, for example, sold under the tradenames Jaguar HP8, Jaguar HP60, Jaguar HP120, Jaguar DC 293 and Jaguar HP 105 by Rhône-Poulenc (Meyhall) or under the name Galactasol 4H4FD2 by Aqualon.

Mention may be made, among the gums of microbial origin, of biopolysaccharide gums, such as scleroglucans or xanthans.

The scleroglucans are represented, for example, by the products sold under the name Actigum CS by Sanofi Bio Industries and in particular Actigum CS 11 and under the name Amigel by Alban Muller International. Other scleroglucans, such as that treated with glyoxal in French patent application No. 2 633 940, can also be used.

The xanthans are represented, for example, by the products sold under the names Keltrol, Keltrol T, Keltrol T F, Keltrol B T, Keltrol R D or Keltrol C G by Nutrasweet Kelco or under the names Rhodicare S or Rhodicare H by Rhodia Chimie.

Mention may be made, among the gums of plant origin, of those resulting from plant exudates, such as gum arabic, ghatti gum, karaya gum, tragacanth gum, carrageenan gum, agar gum and locust bean gum.

Use may also be made of pectins, alginates and starches.

All these compounds are well known to a person skilled in the art and are described in particular in the work by Robert L. Davidson entitled “Handbook of water-soluble gums and resins”, published by McGraw Hill Book Company (1980).

Synthetic Thickeners:

Including amphiphilic polymers, also known as “associative” polymers, comprising at least one fatty chain and of anionic, nonionic, cationic or amphoteric type.

Amphiphilic or “Associative” Polymers:

Mention may be made, among the polymers comprising at least one fatty chain and of anionic type, of:
(I) those comprising at least one hydrophilic unit and at least one allyl ether unit comprising a fatty chain, more particularly those for which the hydrophilic unit is composed of an ethylenic unsaturated anionic monomer, more particularly still of a vinyl carboxylic acid and very particularly of an acrylic acid or a methacrylic acid or the mixtures of these, and for which the allyl ether unit comprising a fatty chain corresponds to the monomer of following formula (XV):
   CH₂═CR′CH₂OBₙR (XV)
   in which R′ denotes H or CH₃, B denotes the ethyleneoxy radical, n is zero or denotes an integer ranging from 1 to 100, and R denotes a hydrocarbonaceous radical chosen from alkyl, arylalkyl, aryl, alkylaryl or cycloalkyl radicals comprising from 8 to 30 carbon atoms, preferably 10 to 24, and more particularly still from 12 to 18 carbon atoms. A more particularly preferred unit of formula (XV) is a unit in which R′ denotes H, n is equal to 10 and R denotes a stearyl (C₁₈) radical.

Anionic amphiphilic polymers of this type are disclosed and prepared, according to an emulsion polymerization process, in patent EP-0 216 479.

Preference is particularly given according to the invention, among these anionic thickening polymers comprising a fatty chain, to the polymers formed from 20 to 60% by weight of acrylic acid and/or methacrylic acid, from 5 to 60% by weight of lower alkyl (meth)acrylates, from 2 to 50% by weight of allyl ether comprising a fatty chain of formula (XV), and from 0 to 1% by weight of a crosslinking agent which is a well known copolymerizable polyethylenic unsaturated monomer, such as diallyl phthalate, allyl (meth)acrylate, divinylbenzene, (poly)ethylene glycol dimethacrylate and methylenebisacrylamide.

Among the latter, preference is very particularly given to crosslinked terpolymers of methacrylic acid, of ethyl acrylate and of polyethylene glycol (10 EO) ether of stearyl alcohol (Steareth 10), in particular those sold by Allied Colloids under the names Salcare SC 80 and Salcare SC90, which are 30% aqueous emulsions of a crosslinked terpolymer of methacrylic acid, of ethyl acrylate and of steareth-10 allyl ether (40/50/10).
(II) those comprising at least one hydrophilic unit of olefinic unsaturated carboxylic acid type and at least one hydrophobic unit of alkyl (C₁₀-C₃₀) ester of unsaturated carboxylic acid type.

Preferably, these polymers are chosen from those for which the hydrophilic unit of olefinic unsaturated carboxylic acid type corresponds to the monomer of following formula (XVI): 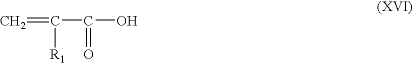
in which R₁ denotes H or CH₃ or C₂H₅, that is to say acrylic acid, methacrylic acid or ethacrylic acid units, and for which the hydrophobic unit of alkyl (C₁₀-C₃₀) ester of unsaturated carboxylic acid type corresponds to the monomer of following formula (XVII): 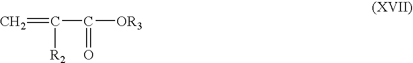
in which R₂ denotes H or CH₃ or C₂H₅ (that is to say, acrylate, methacrylate or ethacrylate units) and preferably H (acrylate units) or CH₃ (methacrylate units), R₃ denoting a C₁₀-C₃₀ and preferably C₁₂-C₂₂ alkyl radical.

Alkyl (C₁₀-C₃₀) esters of unsaturated carboxylic acids in accordance with the invention comprise, for example, lauryl acrylate, stearyl acrylate, decyl acrylate, isodecyl acrylate, dodecyl acrylate, and the corresponding methacrylates, lauryl methacrylate, stearyl methacrylate, decyl methacrylate, isodecyl methacrylate and dodecyl methacrylate.

Anionic polymers of this type are, for example, disclosed and prepared according to patents U.S. Pat. Nos. 3 915 921 and 4 509 949.

Use will more particularly be made, among this type of anionic thickening polymers comprising a fatty chain, of polymers formed from a mixture of monomers comprising:
(i) essentially acrylic acid,
(ii) an ester of formula (XVI) described above and in which R₂ denotes H or CH₃, R₃ denoting an alkyl radical having from 12 to 22 carbon atoms,
(iii) and a crosslinking agent which is a well known copolymerizable polyethylenic unsaturated monomer, such as diallyl phthalate, allyl (meth)acrylate, divinylbenzene, (poly)ethylene glycol dimethacrylate and methylenebisacrylamide.

Use will more particularly be made, among this type of anionic thickening polymers comprising a fatty chain, of those composed of 95 to 60% by weight of acrylic acid (hydrophilic unit), 4 to 40% by weight of C₁₀-C₃₀ alkyl acrylate (hydrophobic unit) and 0 to 6% by weight of crosslinking polymerizable monomer or else those composed of 98 to 96% by weight of acrylic acid (hydrophilic unit), 1 to 4% by weight of C₁₀-C₃₀ alkyl acrylate (hydrophobic unit) and 0.1 to 0.6% by weight of crosslinking polymerizable monomer such as those described above.

Preference is very particularly given according to the present invention, among said polymers above, to the products sold by Goodrich under the tradenames Pemulen TR1, Pemulen TR2 or Carbopol 1382 and more preferably still to Pemulen TR1 and the product sold by S.E.P.P.I.C. under the name Coatex SX.
(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608 by Newphase Technologies.
(IV) acrylic terpolymers comprising:
   (a) approximately 20% to 70% by weight of a carboxylic acid comprising a,pmonoethylenic unsaturation,
   (b) approximately 20 to 80% by weight of a non-surface-active monomer comprising α,β-monoethylenic unsaturation other than (a),
   (c) approximately 0.5 to 60% by weight of a nonionic monourethane which is the reaction product of a monohydric surfactant with a monoisocyanate comprising monoethylenic unsaturation,
   such as those disclosed in patent application EP-A-0 173 109 and more particularly that disclosed in example 3, namely a methacrylic acid/methyl acrylate/dimethyl(meta-isopropenyl)benzyl isocyanate of ethoxylated behenyl alcohol (40 EO) terpolymer as a 25% aqueous dispersion.
(V) copolymers comprising, among their monomers, a carboxylic acid comprising α,β-monoethylenic unsaturation and an ester of a carboxylic acid comprising α,β-monoethylenic unsaturation and of an oxyalkylenated fatty alcohol.

Preferably, these compounds also comprise, as monomer, an ester of a carboxylic acid comprising α,β-monoethylenic unsaturation and of a C₁-C₄ alcohol.

Mention may be made, as example of this type of compound, of Aculyn 22, sold by Röhm & Haas, which is a methacrylic acid/ethyl acrylate/oxyalkylenated stearyl methacrylate terpolymer.

Among the polymers comprising a fatty chain of nonionic type, the choice is preferably made of:
(1) celluloses modified by groups comprising at least one fatty chain; mention may be made, by way of example, of:
   hydroxyethylcelluloses modified by groups comprising at least one fatty chain, such as alkyl, arylalkyl or alkylaryl groups, or their mixtures, and in which the alkyl groups are preferably C₈-C₂₂ groups, such as the product Natrosol Plus Grade 330 CS (C₁₆ alkyls), sold by Aqualon, or the product Bermocoll EHM 100, sold by Berol Nobel,
   those modified by polyalkylene glycol ether of alkylphenol groups, such as the product Amercell Polymer HM-1500 (polyethylene glycol (15) ether of nonylphenol), sold by Amerchol.
(2) hydroxypropylguars modified by groups comprising at least one fatty chain, such as the product Esaflor HM 22 (C₂₂ alkyl chain), sold by Lamberti, or the products RE210-18 (C₁₄ alkyl chain) and RE205-1 (C₂₀ alkyl chain), sold by Rhône-Poulenc.
(3) copolymers of vinylpyrrolidone and of hydrophobic monomers comprising a fatty chain;
mention may be made, by way of example, of:
   the products Antaron V216 or Ganex V216 (vinylpyrrolidone/hexadecene copolymer), sold by I.S.P.
   the products Antaron V220 or Ganex V220 (vinylpyrrolidone/icosene copolymer), sold by I.S.P.
(4) copolymers of C₁-C₆ alkyl methacrylates or acrylates and of amphiphilic monomers comprising at least one fatty chain, such as, for example, the methyl acrylate/oxyethylenated stearyl acrylate copolymer sold by Goldschmidt under the name Antil 208.
(5) copolymers of hydrophilic methacrylates or acrylates and of hydrophobic monomers comprising at least one fatty chain, such as, for example, the polyethylene glycol methacrylate/lauryl methacrylate copolymer.
(6) polyether polyurethanes comprising, in their chain, both hydrophilic blocks of generally polyoxyethylene nature and hydrophobic blocks which can be aliphatic sequences alone and/or cycloaliphatic and/or aromatic sequences.
(7) polymers comprising an aminoplast ether backbone having at least one fatty chain, such as the compounds Pure Thix provided by Sud-Chemie.

Preferably, the polyether polyurethanes comprise at least two hydrocarbonaceous lipophilic chains, having from 6 to 30 carbon atoms, separated by a hydrophilic block, it being possible for the hydrocarbonaceous chains to be pendent chains or chains at the end of a hydrophilic block. In particular, it is possible for one or more pendent chains to be provided. In addition, the polymer can comprise a hydrocarbonaceous chain at one end or at both ends of a hydrophilic block.

The polyether polyurethanes can be multiblock, in particular in the triblock form. The hydrophobic blocks can be at each end of the chain (for example: triblock copolymer comprising a central hydrophilic block) or distributed both at the ends and in the chain (multiblock copolymer, for example). These same polymers can also be graft polymers or star polymers.

The nonionic polyether polyurethanes comprising a fatty chain can be triblock copolymers for which the hydrophilic block is a polyoxyethylene chain comprising from 50 to 1 000 oxyethylene groups. The nonionic polyether polyurethanes comprise a urethane bond between the hydrophilic blocks, hence the name.

By extension, the nonionic polyether polyurethanes comprising a fatty chain also include those for which the hydrophilic blocks are bonded to the lipophilic blocks via other chemical bonds.

Use may also be made, as examples of nonionic polyether polyurethanes comprising a fatty chain which can be used in the invention, also of Rheolate 205 comprising a urea functional group, sold by Rheox, or Rheolates 208, 204 or 212, and Acrysol RM 184, Aculyn 44 and Aculyn 46 from Röhm & Haas [Aculyn 46 is a polycondensate of polyethylene glycol comprising 150 or 180 mol of ethylene oxide, of stearyl alcohol and of methylenebis(4-cyclohexyl isocyanate) (SMDI), at 15% by weight in a matrix of maltodextrin (4%) and of water (81%); Aculyn 44 is a polycondensate of polyethylene glycol comprising 150 or 180 mol of ethylene oxide, of decyl alcohol and of methylenebis(4-cyclohexyl isocyanate) (SMDI), at 35% by weight in a mixture of propylene glycol (39%) and of water (26%)].

Mention may also be made of the product Elfacos T210 comprising a C₁₂₋₁₄ alkyl chain and the product Elfacos T212 comprising a C₁₈ alkyl chain from Akzo.

The product DW 1206B from Röhm & Haas comprising a C₂₀ alkyl chain and comprising a urethane bond, provided at a dry matter content of 20% in water, can also be used.

Use may also be made of solutions or dispersions of these polymers, in particular in water or in an aqueous/alcoholic medium. Mention may be made, by way of example of such polymers, of Rheolate 255, Rheolate 278 and Rheolate 244, sold by Rheox. Use may also be made of the product DW 1206F and DW 1206J, provided by Röhm & Haas.

The polyether polyurethanes which can be used according to the invention are in particular those described in the article by G. Fonnum, J. Bakke and Fk. Hansen, Colloid Polym. Sci., 271, 380-389 (1993).

Mention may in particular be made, among the Polymers comprising a fatty chain and of cationic type, of quaternized cellulose derivatives which are, in particular,
quaternized celluloses modified by groups comprising at least one fatty chain, such as alkyl, arylalkyl or alkylaryl groups comprising at least 8 carbon atoms, or mixtures of these groups,
quaternized hydroxyethylcelluloses modified by groups comprising at least one fatty chain, such as alkyl, arylalkyl or alkylaryl groups comprising at least 8 carbon atoms, or mixtures of these groups.

The alkyl radicals carried by the above quaternized celluloses or hydroxyethylcelluloses preferably comprise from 8 to 30 carbon atoms. The aryl radicals preferably denote the phenyl, benzyl, naphthyl or anthryl groups.

Examples of quaternized alkylhydroxyethyl-celluloses comprising C₈-C₃₀ fatty chains which may be pointed out include the products Quatrisoft LM 200, Quatrisoft LM-X 529-18-A, Quatrisoft LM-X 529-18B (C₁₂ alkyl) and Quatrisoft LM-X 529-8 (C₁₈ alkyl), sold by Amerchol, and the products Crodacel QM, Crodacel QL (C₁₂ alkyl) and Crodacel QS (C,₈ alkyl), sold by Croda.

Polyacrylates comprising aminated side groups, which may or may not be quaternized, have, for example, hydrophobic groups of the Steareth-20 type (polyoxyethylenated (20) stearyl alcohol).

Mention may be made, as examples of polyacrylates comprising aminated side chains, of the polymers 8781-121B or 9492-103 provided by National Starch.

Among the Polymers comprising at least one fatty chain and of amphoteric type, the choice is preferably made of those comprising at least one noncyclic cationic unit. More particularly still, preference is given to those prepared from or comprising 1 to 20 mol % of monomer comprising a fatty chain and preferably 1.5 to 15 mol % and more particularly still 1.5 to 6 mol % , with respect to the total number of moles of monomers.

The amphoteric polymers comprising a fatty chain which are preferred according to the invention comprise, or are prepared by copolymerizing:
1) at least one monomer of formula (Ia) or (Ib): 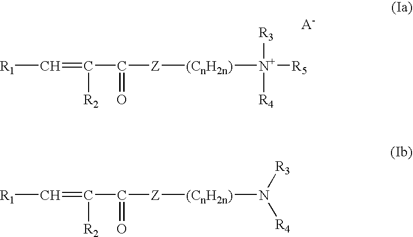
   in which R₁ and R₂, which are identical or different, represent a hydrogen atom or a methyl radical and R₃, R₄ and R₅, which are identical or different, represent a linear or branched alkyl radical having from 1 to 30 carbon atoms,
Z represents an NH group or an oxygen atom,
n is an integer from 2 to 5,
A⁻ is an anion resulting from an organic or inorganic acid, such as a methyl sulfate anion or a halide, such as chloride or bromide;
2) at least one monomer of formula (II)
   R₆—CH═CR₇—COOH (II)
   in which R₆ and R₇, which are identical or different, represent a hydrogen atom or a methyl radical;
   and
3) at least one monomer of formula (III):
   R₆—CH═CR₇—COXR₈ (III)
   in which R₆ and R₇, which are identical or different, represent a hydrogen atom or a methyl radical, X denotes an oxygen or a nitrogen atom and R8 denotes a linear or branched alkyl radical having from 1 to 30 carbon atoms;
at least one of the monomers of formula (Ia), (Ib) or (III) comprising at least one fatty chain.

The monomers of formula (Ia) and (Ib) of the present invention are preferably chosen from the group consisting of:
dimethylaminoethyl methacrylate, dimethylaminoethyl acrylate,
diethylaminoethyl methacrylate, diethylaminoethyl acryate,
dimethylaminopropyl methacrylate, dimethylaminopropyl acrylate,
dimethylaminopropylmethacrylamide, dimethylaminopropylacrylamide, these monomers optionally being quaternized, for example with a C₁-C₄ alkyl halide or a di(C₁-C₄ alkyl) sulfate.

More particularly, the monomer of formula (la) is chosen from acrylamidopropyltrimethylammonium chloride and methacrylamidopropyltrimethyl ammonium chloride.

The monomers of formula (II) of the present invention are preferably chosen from the group consisting of acrylic acid, methacrylic acid, crotonic acid and 2-methylcrotonic acid. More particularly, the monomer of formula (II) is acrylic acid.

The monomers of formula (III) of the present invention are preferably chosen from the group consisting of C₁₂-C₂₂ and more particularly C₁₆-C₁₈ alkyl acrylates or methacrylates.

The monomers constituting the amphoteric polymers comprising a fatty chain of the invention are preferably already neutralized and/or quaternized.

The ratio of the number of cationic charges/anionic charges is preferably equal to approximately 1.

The amphoteric polymers comprising a fatty chain according to the invention preferably comprise from 1 to 10 mol % of the monomer comprising a fatty chain (monomer of formula (Ia), (Ib) or (III)) and preferably from 1.5 to 6 mol % .

The weight-average molecular weights of the amphoteric polymers comprising a fatty chain according to the invention can vary from 500 to 50,000,000 and are preferably between 10,000 and 5,000,000.

The amphoteric polymers comprising a fatty chain according to the invention can also comprise other monomers, such as nonionic monomers and in particular such as C₁-C₄ alkyl acrylates or methacrylates.

Amphoteric polymers comprising a fatty chain according to the invention are, for example, disclosed and prepared in patent application WO 98/44012.

Preference is given, among the amphoteric polymers comprising a fatty chain according to the invention, to acrylic acid/(meth)acrylamidopropyltrimethylammonium chloride/stearyl methacrylate copolymers.

The bleaching composition in accordance with the invention can also include various adjuvants conventionally used in compositions for bleaching the hair.

Adjuvants:

Among these adjuvants, surface-active agents can be present and can be chosen without distinction, alone or as mixtures, from anionic, amphoteric, nonionic, zwitterionic and cationic surfactants.

The surfactants which are suitable for implementing the present invention are in particular the following:

(i) Anionic Surfactant(s):

Mention may in particular be made, as example of anionic surfactants which can be used, alone or [lacuna] mixtures, in the context of the present invention, of (nonlimiting list) the salts (in particular alkali metal, especially sodium, salts, ammonium salts, amine salts, aminoalcohol salts or magnesium salts) of the following compounds: alkyl sulfates, alkyl ether sulfates, alkylamido ether sulfates, alkylarylpolyether sulfates, monoglyceride sulfates; alkylsulfonates, alkyl phosphates, alkylamidesulfonates, alkylarylsulfonates, α-olefinsulfonates, paraffinsulfonates; (C₆-C₂₄)alkyl sulfosuccinates, (C₆-C₂₄)alkyl ether sulfosuccinates, (C₆-C₂₄)alkylamidesulfosuccinates; (C₆-C₂₄)alkyl sulfoacetates; (C₆-C₂₄)acylsarcosinates and (C₆-C₂₄)acylglutamates. Use may also be made of the esters of carboxylic (C₆-C₂₄)alkylpolyglycosides, such as alkylglucoside citrates, alkylpolyglycoside tartrate and alkylpolyglycoside sulfosuccinates, alkyl sulfosuccinamates; acylisethionates and N-acyltaurates, the alkyl or acyl radical of all these various compounds preferably comprising from 12 to 20 carbon atoms and the aryl radical preferably denoting a phenyl or benzyl group. Mention may also be made, among anionic surfactants which can also be used, of the salts of fatty acids, such as the salts of oleic acid, ricinoleic acid, palmitic acid, stearic acid, coconut oil acid or hydrogenated coconut oil acid; or acyllactylates, the acyl radical of which comprises 8 to 20 carbon atoms. Use may also be made of alkyl-D-galactosideuronic acids and their salts, (C₆-C₂₄)alkyl polyoxyalkylenated ether carboxylic acids, (C₆-C₂₄)alkylaryl polyoxyalkylenated ether carboxylic acids, (C₆-C₂₄)alkylamido polyoxyalkylenated ether carboxylic acids and their salts, in particular those comprising from 2 to 50 alkylene oxide, in particular ethylene oxide, groups, and their mixtures.

(ii) Nonionic Surfactant(s):

The nonionic surface-active agents themselves are also compounds which are well known per se (in this respect see in particular the “Handbook of Surfactants” by M. R. Porter, published by Blackie & Son (Glasgow and London), 1991, pp. 116-178) and, in the context of the present invention, their nature does not assume any critical character. They can thus be chosen especially from (nonlimiting list) fatty alcohols, a-diols, alkylphenols or acids which are polyethoxylated or polypropoxylated, having a fatty chain comprising, for example, 8 to 18 carbon atoms, it being possible for the number of ethylene oxide or propylene oxide groups to range especially from 2 to 50. Mention may also be made of the copolymers of ethylene and propylene oxide and the condensates of ethylene and propylene oxide with fatty alcohols; the polyethoxylated fatty amides preferably having from 2 to 30 mol of ethylene oxide, the polyglycerolated fatty amides on average comprising 1 to 5 glycerol groups and in particular 1.5 to 4; the polyethoxylated fatty amines preferably having 2 to 30 mol of ethylene oxide; the oxyethylenated esters of sorbitan fatty acids having from 2 to 30 mol of ethylene oxide; the sucrose esters of fatty acids, the polyethylene glycol esters of fatty acids, alkylpolyglycosides, the N-alkylglucamine derivatives, or amine oxides, such as the oxides of (C₁₀-C₁₄)alkylamines or the N-acylaminopropylmorpholine oxides. It will be noted that alkylpolyglycosides constitute nonionic surfactants which come particularly well within the scope of the present invention.

(iii) Amphoteric or Zwitterionic Surfactant(s):

The amphoteric or zwitterionic surface-active agents, the nature of which does not assume any critical character in the context of the present invention, may be especially (nonlimiting list) derivatives of aliphatic secondary or tertiary amines in which the aliphatic radical is a linear or branched chain comprising 8 to 18 carbon atoms and comprising at least one water-solubilizing anionic group (for example carboxylate, sulfonate, sulfate, phosphate or phosphonate); (C₈-C₂₀)alkyl betaines, sulfobetaines, (C₈-C₂₀)alkyl amido(C₁-C₆)alkyl betaines or (C₈-C₂₀)alkyl amido(C₁-C₆)alkyl sulfobetaines may further be mentioned.

Mention may be made, among the amine derivatives, of the products sold under the name Miranol, as disclosed in patents U.S. Pat. No. 2,528,378 and U.S. Pat. No. 2,781,354 and classified in the CTFA dictionary, 3rd edition, 1982, under the names Amphocarboxyglycinates and Amphocarboxypropionates with the respective structures:
R₃₄—CONHCH₂CH₂—N(R₃₅)(R₃₆)(CH₂COO⁻)
in which: R₃₄ denotes an alkyl radical of an acid R₃₄—COOH present in hydrolyzed coconut oil, a heptyl, nonyl or undecyl radical, R₃₅ denotes a β-hydroxyethyl group and R₃₆ a carboxymethyl group;
and
R₃₄′—CONHCH₂CH₂—N(B)(C)
in which:
B represents —CH₂CH₂OX′, C represents —(CH₂)_{z}—Y′, with z=1 or 2,
X′ denotes the —CH₂CH₂—COOH group or a hydrogen atom
Y′ denotes —COOH or the radical —CH₂—CHOH—SO₃H
R₃₄′ denotes an alkyl radical of an acid R₃₇—COOH present in coconut oil or in hydrolyzed linseed oil, an alkyl radical, in particular C₇, C₉, C₁₁ or C₁₃, a C₁₇ alkyl radical and its iso form, or an unsaturated C₁₇ radical.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid and Cocoamphodipropionic acid.

Mention may be made, by way of example, of the cocoamphodiacetate sold under the tradename Miranol® C2M concentré by Rhodia Chimie.

(iv) Cationic Surfactants:

Mention may be made, among the cationic surfactants, of in particular (nonlimiting list): the salts of optionally polyoxyalkylenated primary, secondary or tertiary fatty amines; quaternary ammonium salts, such as tetraalkylammonium, alkylamidoalkyltrialkylammonium, trialkylbenzylammonium, trialkylhydroxyalkyl ammonium or alkylpyridinium chlorides or bromides; imidazoline derivatives; or amine oxides with a cationic nature.

The amounts of surface-active agents present in the ready-to-use composition according to the invention can vary from 0.01 to 40% and preferably from 0.1 to 30% of the total weight of the composition.

Other adjuvants can also be present and among them nonionic, anionic, amphoteric, zwitterionic and cationic conditioning polymers, or their mixtures, and preferably cationic or amphoteric substantive polymers.

The substantive nature (that is to say, the ability to be deposited on the hair) of the polymers used in accordance with the invention is conventionally determined by means of the test described by Richard J. Crawford, Journal of the Society of Cosmetic Chemists, 1980, 31(5), pages 273 to 278 (development by Acid Red 80 dye).

Cationic Substantive Polymers:

Within the meaning of the present invention, the expression “cationic polymer” denotes any polymer comprising cationic groups and/or groups which can be ionized to cationic groups.

The cationic polymers which can be used in accordance with the present invention can be chosen from all those already known per se as improving the cosmetic properties of the hair, namely, in particular, those disclosed in patent application EP-A-337 354 and in French patents FR-2 270 846, 2 383 660, 2 598 611, 2 470 596 and 2 519 863.

The preferred cationic polymers are chosen from those which comprise units comprising primary, secondary, tertiary and/or quaternary amine groups which can either form part of the main polymer chain or be carried by a side substituent directly connected to the main chain.

The cationic polymers used generally have a number-average molecular mass of between 500 and 5×10⁶ approximately and preferably of between 10³ and 3×10⁶ approximately.

Mention may more particularly be made, among cationic polymers, of the polymers of the polyamine, polyaminoamide and poly(quaternary ammonium) type.

These are known products. They are disclosed in particular in French patents No. 2 505 348 or 2 542 997. Mention may be made, among said polymers, of:
(1) homopolymers or copolymers derived from acrylic or methacrylic esters or amides comprising at least one of the units of following formulae (I), (II), (III) or (IV): 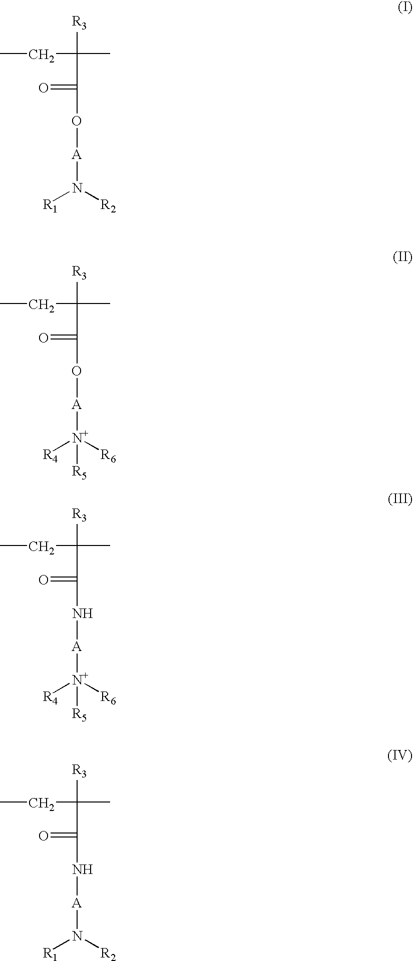
   in which:
R₃, which are identical or different, denote a hydrogen atom or a CH₃ radical;
A, which are identical or different, represent a linear or branched alkyl group comprising 1 to 6 carbon atoms, preferably 2 or 3 carbon atoms, or a hydroxyalkyl group comprising 1 to 4 carbon atoms;
R₄, R₅ and R₆, which are identical or different, represent an alkyl group having from 1 to 18 carbon atoms or a benzyl radical and preferably an alkyl group having from I to 6 carbon atoms;
R₁ and R₂, which are identical or different, represent hydrogen or an alkyl group having from 1 to 6 carbon atoms and preferably methyl or ethyl;
X denotes an anion derived from an inorganic or organic acid, such as a methyl sulfate anion or a halide, such as chloride or bromide.

The polymers of family (1) can additionally comprise one or more units deriving from comonomers which can be chosen from the family of the acrylamides, methacrylamides, diacetone acrylamides, acrylamides and methacrylamides substituted on the nitrogen by lower (C₁-C₄) alkyls, acrylic or methacrylic acids or their esters, vinyllactams, such as vinylpyrrolidone or vinylcaprolactam, or vinyl esters.

Thus, mention may be made, among these polymers of family (1), of:
copolymers of acrylamide and of dimethylaminoethyl methacrylate which is quaternized with dimethyl sulfate or with a dimethyl halide, such as that sold under the name Hercofloc by Hercules,
copolymers of acrylamide and of methacryloyloxyethyltrimethylammonium chloride, disclosed, for example, in patent application EP-A-080 976 and sold under the name Bina Quat P 100 by Ciba-Geigy,
the copolymer of acrylamide and of methacryloyloxyethyltrimethylammonium methyl sulfate sold under the name Reten by Hercules,
vinylpyrrolidone/dialkylaminoalkyl acrylate or methacrylate copolymers, which may or may not be quaternized, such as the products sold under the name “Gafquat” by ISP, like for example “Gafquat 734” or “Gafquat 755”, or the products named “Copolymer 845, 958 and 937”. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by ISP,
vinylpyrrolidone/methacrylamidopropyldimethylamine copolymer, sold in particular under the name Styleze CC 10 by ISP,
and vinylpyrrolidone/quaternized dimethylaminopropylmethacrylamide copolymers, such as the product sold under the name “Gafquat HS 100” by ISP.
(2) Cellulose ether derivatives comprising quaternary ammonium groups disclosed in French patent 1 492 597 and in particular the polymers sold under the names “JR” (JR 400, JR 125, JR 30M) or “LR” (LR 400, LR 30M) by Union Carbide Corporation. These polymers are also defined in the CTFA dictionary as quaternary ammonium of hydroxyethylcellulose having reacted with an epoxide substituted by a trimethylammonium group.
(3) Cationic cellulose derivatives, such as the copolymers of cellulose or the cellulose derivatives grafted with a water-soluble quaternary ammonium monomer and disclosed in particular in patent U.S. Pat. No. 4 131 576, such as hydroxyalkyl celluloses, for example hydroxymethyl, hydroxyethyl or hydroxypropyl celluloses, grafted in particular with a methacryloylethyltrimethylammonium, methacrylmidopropyltrimethylammonium or dimethyldiallylammonium salt.

The marketed products corresponding to this definition are more particularly the products sold under the name “Celquat L 200” and “Celquat H 100” by National Starch.
(4) The cationic polysaccharides disclosed more particularly in patents U.S. Pat. Nos. 3,589,578 and 4,031,307, such as guar gums comprising trialkylammonium cationic groups. Use is made, for example, of guar gums modified by a 2,3-epoxypropyltrimethylammonium salt (e.g. chloride).

Such products are sold in particular under the tradenames of Jaguar C13S, Jaguar C 15, Jaguar C 17 or Jaguar C162 by Meyhall.
(5) Polymers composed of piperazinyl units and of divalent, straight- or branched-chain alkylene or hydroxyalkylene radicals, optionally interrupted by oxygen, sulfur or nitrogen atoms or by aromatic or heterocyclic rings, as well as the oxidation and/or quaternization products of these polymers. Such polymers are disclosed in particular in French patents 2 162 025 and 2 280 361.
(6) Water-soluble polyaminoamides prepared in particular by polycondensation of an acidic compound with a polyamine; these polyaminoamides can be crosslinked by an epihalohydrin, a diepoxide, a dianhydride, an unsaturated dianhydride, a bisunsaturated derivative, a bishalohydrin, a bisazetidinium, a bishaloacyidiamine or an alkyl bishalide or alternatively by an oligomer resulting from the reaction of a bifunctional compound reactive with respect to a bishalohydrin, a bisazetidinium, a bishaloacyidiamine, an alkyl bishalide, an epilhalohydrin, a diepoxide or a bisunsaturated derivative; the crosslinking agent being used in proportions ranging from 0.025 to 0.35 mol per amine group of the polyaminoamide; these polyaminoamides can be alkylated or, if they comprise one or more tertiary amine functional groups, quaternized. Such polymers are disclosed in particular in French patents 2 252 840 and 2 368 508.
(7) Polyaminoamide derivatives resulting from the condensation of polyalkylenepolyamines with polycarboxylic acids, followed by an alkylation by bifunctional agents. Mention may be made, for example, of adipic acid/diakylaminohydroxyalkyldialoylenetriamine polymers in which the alkyl radical comprises from 1 to 4 carbon atoms and preferably denotes methyl, ethyl or propyl. Such polymers are disclosed in particular in French patent 1 583 363.

Mention may more particularly be made, among these derivatives, of the adipic acid/dimethylaminohydroxypropyl/diethylenetriamine polymers sold under the name “Cartaretine F, F4 or F8” by Sandoz.
(8) Polymers obtained by reaction of a polyalkylenepolyamine comprising two primary amine groups and at least one secondary amine group with a dicarboxylic acid chosen from diglycolic acid and saturated aliphatic dicarboxylic acids having from 3 to 8 carbon atoms. The molar ratio of polyalkylenepolylamine to dicarboxylic acid being between 0.8:1 and 1.4:1; the polyaminoamide resulting therefrom being reacted with epichlorohydrin in a molar ratio of epichlorohydrin in relation to the secondary amine group of the polyaminoamide of between 0.5:1 and 1.8:1. Such polymers are disclosed in particular in U.S. Pat. Nos. 3,227,615 and 2,961,347.

Polymers of this type are in particular sold under the name “Hercosett 57” by Hercules Inc. or else under the name of “PD 170” or “Delsette 101” by Hercules in the case of the adipic acid/epoxypropyl/diethylenetriamine copolymer.
(9) Cyclopolymers of alkyldiallylamine or of dialkyldiallylammonium, such as the homopolymers or copolymers comprising, as main constituent of the chain, units corresponding to the formulae (V) or (VI): 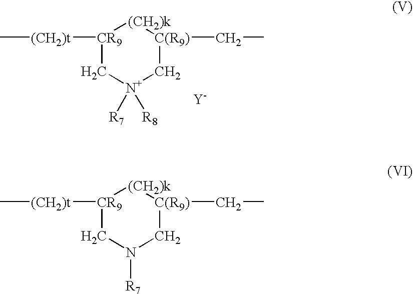
   in which formulae k and t are equal to 0 or 1, the sum k+t being equal to 1; R₉ denotes a hydrogen atom or a methyl radical; R₇ and R₈, independently of one another, denote an alkyl group having from 1 to 22 carbon atoms, a hydroxyalkyl group in which the alkyl group preferably has 1 to 5 carbon atoms or a lower amidoalkyl (C₁-C₄) group or R₇ and R₈ can denote, jointly with the nitrogen atom to which they are attached, heterocyclic group, such as piperidinyl or morpholinyl; R₇ and R₈, independently of one another, preferably denote an alkyl group having from 1 to 4 carbon atoms; Y⁻ is an anion, such as bromide, chloride, acetate, borate, citrate, tartrate, bisulfate, bisulfite, sulfate or phosphate. These polymers are disclosed in particular in French patent 2 080 759 and in its certificate of addition 2 190 406.

Mention may more particularly be made, among the polymers defined above, of the homopolymer of dimethyldiallylammonium chloride sold under the name “Merquat 100” by Calgon (and its homologs of low weight-average molecular mass) and of the copolymers of diallyldimethylammonium chloride and of acrylamide sold under the name “Merquat 550”.
(10) The quaternary diammonium polymer comprising repeat units corresponding to the formula: 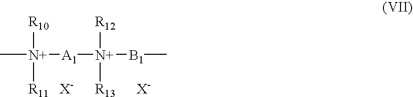
   in which formula (VII):
R₁₀, R₁₁, R₁₂ and R₁₃, which are identical or different, represent aliphatic, alicyclic or arylaliphatic radicals comprising from 1 to 20 carbon atoms or lower aliphatic hydroxyalkyl radicals or R₁₀, R₁₁, R₁₂ and R₁₃, together or separately, form, with the nitrogen atoms to which they are attached, heterocycles optionally comprising a second heteroatom other than nitrogen or else R₁₀, R₁₁, R₁₂ and R₁₃ represent a linear or branched C₁-C₆ alkyl radical substituted by a nitrile, ester, acyl, amide or —CO—O—R₁₄-D or —CO—NH—R₁₄-D group, where R₁₄ is an alkylene and D a quaternary ammonium group;
A₁ and B₁ represent polymethylene groups comprising from 2 to 20 carbon atoms which can be linear or branched and saturated or unsaturated, and which can comprise, bonded to or inserted into the main chain, one or more aromatic rings or one or more oxygen or sulfur atoms or sulfoxide, sulfone, disulfide, amino, alkylamino, hydroxyl, quaternary ammonium, ureido, amide or ester groups, and
X⁻ denotes an anion derived from an inorganic or organic acid;
A₁, R₁₀ and R₁₂ can form, with the two nitrogen atoms to which they are attached, a piperazine ring; in addition, if A₁ denotes a linear or branched, saturated or unsaturated alkylene or hydroxyalkylene radical, B₁ can also denote a —(CH₂)ₙ—CO-D-OC—(CH₂)ₙ— group in which D denotes:
   a) a glycol residue of formula: -O-Z-O-, where Z denotes a linear or branched hydrocarbonaceous radical or a group corresponding to one of the following formulae:
      —(CH₂—CH₂—O)ₓ—CH₂—CH₂—
      [CH₂—CH(CH₃)—O]_{y}—CH₂—CH(CH₃)—
      where x and y denote an integer from 1 to 4, representing a defined and unique degree of polymerization, or any number from 1 to 4 representing a mean degree of polymerization;
   b) a bissecondary diamine residue, such as a piperazine derivative;
   c) a bisprimary diamine residue of formula: —NH—Y—NH—, where Y denotes a linear or branched hydrocarbonaceous radical or else the divalent radical
      —CH₂—CH₂—S—S—CH₂—CH₂—;
   d) a ureylene group of formula: —NH—CO—NH—.

Preferably, X⁻ is an anion, such as chloride or bromide.

These polymers have a number-average molecular mass generally of between 1,000 and 100,000.

Polymers of this type are disclosed in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may more particularly be made of the polymers which are composed of repeat units corresponding to the following formula (VIII): 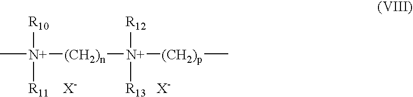
in which R₁₀, R₁₁, R₁₂ and R₁₃, which are identical or different, denote an alkyl or hydroxyalkyl radical having from 1 to 4 carbon atoms approximately, n and p are integers varying from 2 to 20 approximately and X⁻ is an anion derived from an inorganic or organic acid.
(11) Polymers of poly(quaternary ammonium) composed of repeat units of formula (IX): 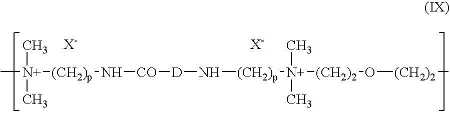
   in which p denotes an integer varying from 1 to 6 approximately, D may not exist or may represent a —(CH₂)ᵣ—CO— group in which r denotes a number equal to 4 or to 7, and X⁻ is an anion.

Such polymers can be prepared according to the processes disclosed in U.S. Pat. Nos. 4,157,388, 4,702,906 and 4,719,282. They are disclosed in particular in patent application EP-A-1 22 324.

Among them, mention may be made, for example, of the products “Mirapol A 15”, “Mirapol AD1”, “Mirapol AZ1” and “Mirapol 175”, sold by Miranol.
(12) Quaternary polymers of vinylpyrrolidone and of vinylimidazole, such as, for example, the products sold under the names Luviquat FC 905, FC 550 and FC 370 by BASF.
(13) Polyamines, such as Polyquart H sold by Henkel, referenced under the name of “Polyethylene Glycol (15) Tallow Polyamine” in the CTFA dictionary.
(14) Crosslinked polymers of methacryloyloxy(C₁-C₄)alkyltri(C₁-C₄)alkylammonium salts, such as the polymers obtained by homopolymerization of dimethylaminoethyl methacrylate quaternized by methyl chloride or by copolymerization of acrylamide with dimethylaminoethyl methacrylate quaternized by methyl chloride, the homo- or copolymerization being followed by a crosslinking by a compound possessing olefinic unsaturation, in particular methylenebisacrylamide. Use may more particularly be made of a crosslinked acrylamide/methacryloyloxyethyltrimethylammonium chloride (20/80 by weight) copolymer in the form of a dispersion comprising 50% by weight of said copolymer in mineral oil. This dispersion is sold under the name of “Salcare® SC 92” by Allied Colloids. Use may also be made of a crosslinked homopolymer of methacryloyloxyethyltrimethylammonium chloride comprising approximately 50% by weight of the homopolymer in mineral oil or in a liquid ester. These dispersions are sold under the names of “Salcare® SC 95” and “Salcare® SC 96” by Allied Colloids.

Other cationic polymers which can be used in the context of the invention are polyalkyleneimines, in particular polyethyleneimines, polymers comprising vinylpyridine or vinylpyridinium units, condensates of polyamines and of epichlorohydrin, quaternary polyureylenes and chitin derivatives.

Among all the cationic polymers capable of being used in the context of the present invention, preference is given to the use of the polymers from families (1), (9), (10), (11) and (14) and more preferably still the polymers with the repeat units of following formulae (W) and (U): 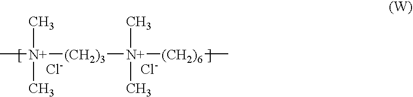
and in particular those for which the molecular weight, determined by gel permeation chromatography, is between 9,500 and 9,900; 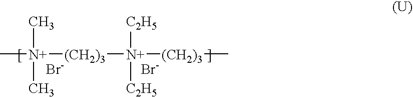
and in particular those for which the molecular weight, determined by gel permeation chromatography, is approximately 1,200.

The concentration of cationic substantive polymer in the composition according to the present invention can vary from 0.01 to 10% by weight with respect to the total weight of the composition, preferably from 0.05 to 5% and more preferably still from 0.1 to 3%.

Amphoteric Substantive Polymers:

The amphoteric polymers which can be used in accordance with the present invention can be chosen from polymers comprising K and M units distributed randomly in the polymer chain, where K denotes a unit deriving from a monomer comprising at least one basic nitrogen atom and M denotes a unit deriving from an acidic monomer comprising one or more carboxyl or sulfo groups or else K and M can denote groups deriving from zwitterionic carboxybetaine or sulfobetaine monomers;
K and M can also denote a cationic polymer chain comprising primary, secondary, tertiary or quaternary amine groups, in which chain at least one of the amine groups carries a carboxyl or sulfo group connected via a hydrocarbon radical, or else K and M form part of a chain of a polymer comprising an α,β-dicarboxyethylene unit, one of the carboxyl groups of which has been reacted with a polyamine comprising one or more primary or secondary amine groups.

The more particularly preferred amphoteric polymers corresponding to the definition given above are chosen from the following polymers:
(1) Polymers resulting from the copolymerization of a monomer derived from a vinyl compound carrying a carboxyl group, such as more particularly acrylic acid, methacrylic acid, maleic acid or α-chloroacrylic acid, and of a basic monomer derived from a substituted vinyl compound comprising at least one basic atom, such as more particularly dialkylaminoalkyl methacrylate and acrylate or dialkylaminoalkylmethacrylamide and -acrylamide. Such compounds are disclosed in U.S. Pat. No. 3,836,537. Mention may also be made of the sodium acrylate/acrylamidopropyltrimethylammonium chloride copolymer sold under the name Polyquart KE 3033 by Henkel.

The vinyl compound can also be a dialkyldiallylammonium salt, such as dimethyldiallylammonium chloride. Copolymers of acrylic acid and of the latter monomer are provided under the names Merquat 280, Merquat 295 and Merquat Plus 3330 by Calgon.
(2) Polymers comprising units deriving:
   a) from at least one monomer chosen from acrylamides or methacrylamides substituted on the nitrogen by an alkyl radical,
   b) from at least one acidic comonomer comprising one or more reactive carboxyl groups, and
   c) from at least one basic comonomer, such as esters comprising primary, secondary, tertiary and quaternary amine substituents of acrylic and methacrylic acids and the quaternization product of dimethylaminoethyl methacrylate with dimethyl or diethyl sulfate.

The more particularly preferred N-substituted acrylamides or methacrylamides according to the invention are the groups in which the alkyl radicals comprise from 2 to 12 carbon atoms and more particularly N-ethylacrylamide, N-tert-butylacrylamide, N-tert-octylacrylamide, N-octylacrylamide, N-decylacrylamide or N-dodecylacrylamide, and the corresponding methacrylamides.

The acidic comonomers are more particularly chosen from acrylic, methacrylic, crotonic, itaconic, maleic or fumaric acids and alkyl monoesters having 1 to 4 carbon atoms of maleic or fumaric acids or anhydrides.

The preferred basic comonomers are aminoethyl, butylaminoethyl, N,N′-dimethylaminoethyl or N-tert-butylaminoethyl methacrylates.

Use is particularly made of the copolymers for which the CTFA name (4th Ed., 1991) is Octylacrylamide/acrylates/butylaminoethyl methacrylate copolymer, such as the products sold under the name Amphomer or Lovocryl 47 by National Starch.
(3) Partially or completely alkylated and crosslinked polyaminoamides deriving from polyaminoamides of general formula:
   —[—CO—R₁₈—CO-Z-]— (X)
   in which R₁₉ represents a divalent radical derived from a saturated dicarboxylic acid, from an aliphatic mono- or dicarboxylic acid comprising an ethylenic double bond, from an ester of a lower alkanol having 1 to 6 carbon atoms of these acids, or from a radical deriving from the addition of any one of said acids with a bisprimary or bissecondary amine, and Z denotes a radical of a bisprimary, mono-or bissecondary polyalkylenepolyamine and preferably represents:
a) in the proportions of 60 to 100 mol % , the radical 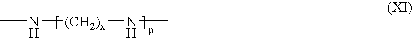
   where x=2 and p=2 or 3, or else x=3 and p=2 this radical deriving from diethylenetriamine, triethylenetetraamine or dipropylenetriamine;
b) in the proportions of 0 to 40 mol % , the above radical (XI), in which x=2 and p=1 and which derives from ethylenediamine, or the radical deriving from piperazine: 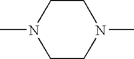
c) in the proportions of 0 to 20 mol % , the radical —NH—(CH₂)₆—NH— deriving from hexamethylene-diamine, these polyaminoamines being crosslinked by addition of a bifunctional crosslinking agent chosen from epihalohydrins, diepoxides, dianhydrides or bisunsaturated derivatives, by means of 0.025 to 0.35mol of crosslinking agent per amine group of the polyaminoamide, and alkylated by reaction with acrylic acid, chloroacetic acid or an alkanesultone or their salts.

The saturated carboxylic acids are preferably chosen from acids having 6 to 10 carbon atoms, such as adipic, 2,2,4-trimethyladipic and 2,4,4-trimethyladipic, or terephthalic acid, and the acids comprising an ethylenic double bond, such as, for example, acrylic, methacrylic or itaconic acids.

The alkanesultones used in the alkylation are preferably propane- or butanesultone and the salts of the alkylating agents are preferably the sodium or potassium salts.
(4) Polymers comprising zwitterionic units of formula: 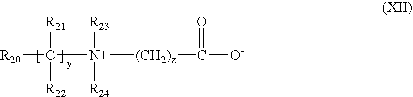
   in which R₂₀ denotes a polymerizable unsaturated group, such as an acrylate, methacrylate, acrylamide or methacrylamide group, y and z represent an integer from 1 to 3, R₂₁ and R₂₂ represent a hydrogen atom or methyl, ethyl or propyl, and R₂₃ and R₂₄ represent a hydrogen atom or an alkyl radical such that the sum of the carbon atoms in R₂₃ and R₂₄ does not exceed 10.

The polymers comprising such units can also comprise units derived from non-zwitterionic monomers, such as dimethyl- or diethylaminoethyl acrylate or methacrylate or alkyl acrylates or methacrylates, acrylamides or methacrylamides, or vinyl acetate.

Mention may be made, by way of example, of butyl methacrylate/dimethylcarboxymethylammonioethyl methacrylate copolymer, such as the product sold under the name Diaformer Z301 by Sandoz.
(5) polymers derived from chitosan comprising monomer units corresponding to the following formulae (XIII), (XIV) and (XV): 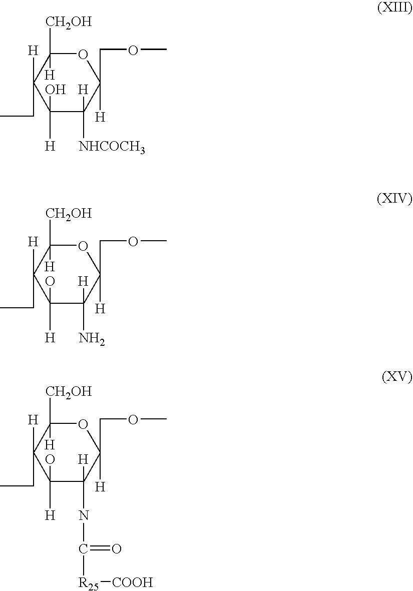
   the unit (XIII) being present in proportions of between 0 and 30%, the unit (XIV) in proportions of between 5 and 50% and the unit (XV) in proportions of between 30 and 90%, it being understood that, in this unit (XV), R₂₅ represents a radical of formula: 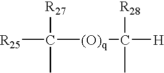
   in which q denotes zero or 1;
if q=0, R₂₆, R₂₇ and R₂₈, which are identical or different, each represent a hydrogen atom, a methyl, hydroxyl, acetoxy or amino residue, a monoalkylamine residue or a dialkylamine residue, optionally interrupted by one or more nitrogen atoms and/or optionally substituted by one or more amine, hydroxyl, carboxyl, alkylthio or sulfo groups, or an alkylthio residue in which the alkyl group carries an amino residue, at least one of the R₂₆, R₂₇ and R₂₈ radicals being, in this case, a hydrogen atom;
or, if q=1, R₂₆, R₂₇ and R₂₈ each represent a hydrogen atom, and the salts formed by these compounds with bases or acids.

Polymers of this type which are more particularly preferred comprise from 0 to 20% by weight of units (XIII), from 40 to 50% by weight of units (XIV) and from 40 to 50% by weight of units (XV) in which R₂₅ denotes the —CH₂—CH₂— radical.
(6) Polymers derived from the N-carboxyalkylation of chitosan, such as the N-(carboxymethyl)chitosan or the N-(carboxybutyl)chitosan sold under the name “Evalsan” by Jan Dekker.
(7) Polymers corresponding to the general formula (XI), such as those disclosed, for example, in French patent 1 400 366: 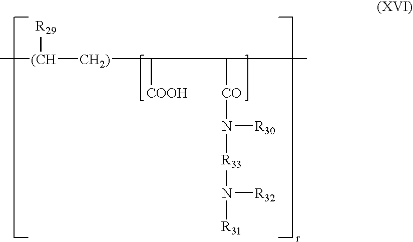
   in which R₂₉ represents a hydrogen atom or a CH₃O, CH₃CH₂O or phenyl radical, R₃₀ denotes hydrogen or a lower alkyl radical, such as methyl or ethyl, R₃₁ denotes hydrogen or a lower alkyl radical, such as methyl or ethyl, and R₃₂ denotes a lower alkyl radical, such as methyl or ethyl, or a radical corresponding to the formula: —R₃₃—N(R₃₁)₂, R₃₃ representing a —CH₂—CH₂—, —CH₂—CH₂—CH₂— or —CH₂—CH(CH₃)— group and R₃₁ having the meanings mentioned above, as well as the higher homologs of these radicals comprising up to 6 carbon atoms; r is such that the molecular weight is between 500 and 6,000,000 and preferably between 1,000 and 1,000,000.
(8) Amphoteric polymers of the -D-X-D-X-type chosen from:
   a) polymers obtained by reaction of chloroacetic acid or sodium chloroacetate with compounds comprising at least one unit of formula:
      -D-X-D-X-D- (XVII)
      where D denotes a radical 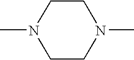
      and X denotes the symbol E or E′, E or E′, which are identical or different, denote a bivalent radical which is a straight- or branched-chain alkylene radical comprising up to 7 carbon atoms in the main chain which is unsubstituted or substituted by hydroxyl groups and which can additionally comprise oxygen, nitrogen or sulfur atoms or 1 to 3 aromatic and/or heterocyclic rings; the oxygen, nitrogen and sulfur atoms being present in the form of ether, thioether, sulfoxide, sulfone, sulfonium, alkylamine or alkenylamine groups or hydroxyl, benzylamine, amine oxide, quaternary ammonium, amide, imide, alcohol, ester and/or urethane groups;
   b) polymers of formula:
      -D-X-D-X- (XVIII)
      where D denotes a radical 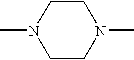
      and X denotes the symbol E or E′ and E′ at least once, E having the meaning indicated above and E′ is a bivalent radical which is a straight- or branched-chain alkylene radical having up to 7 carbon atoms in the main chain which is substituted or unsubstituted by one or more hydroxyl radicals and which comprises one or more nitrogen atoms, the nitrogen atom being substituted by an alkyl chain optionally interrupted by an oxygen atom and necessarily comprising one or more carboxyl functional groups or one or more hydroxyl functional groups and betainized by reaction with chloroacetic acid or sodium chloroacetate.
(9) (C₁-C₅)Alkyl vinyl ether/maleic anhydride copolymers partially modified by semiamidation with an N,N-dialkylaminoalkylamine, such as N,N-dimethylaminopropylamine, or by serniamidation with an N,N-dialkanolamine. These copolymers can also comprise other vinyl comonomers, such as vinylcaprolactam.

The amphoteric polymers which are particularly preferred according to the invention are those of family (1).

According to the invention, the amphoteric substantive polymer or polymers can represent from 0.01% to 10% by weight, preferably from 0.05% to 5% by weight and more preferably still from 0.1% to 3% by weight of the total weight of the composition.

Other adjuvants, such as penetrating agents, sequestering agents, fragrances, dispersing agents, volatile or nonvolatile and modified or unmodified silicones, film-forming agents, ceramides, preservatives, opacifying agents, mineral or vegetable oils, waxes or vitamins, can also be present in the bleaching composition according to the invention.

Of course, a person skilled in the art will take care to choose this or these optional additional compounds so that the advantageous properties intrinsically attached to the bleaching composition in accordance with the invention are not, or not substantially, detrimentally affected by the envisaged addition or additions.

The composition in accordance with the invention can be provided in various forms, such as in the form of solutions, of emulsions, of creams or of gels, optionally pressurized in the form of mousses, or in any other form appropriate for carrying out bleaching of keratinous fibers and in particular of human hair.

It can be obtained by extemporaneous mixing at the time of use either (a) of an anhydrous composition comprising at least one bleaching system as defined above and of an aqueous composition at an acidic pH, or (b) of two aqueous compositions, one of which comprises at least one sulfinic acid or one of its salts at an acidic or alkaline pH and the other of which comprises at least one α-oxocarboxylic acid or one of its salts at an acidic pH, or (c) of an anhydrous composition comprising at least one sulfinic acid or one of its salts and of an aqueous composition comprising at least one α-oxocarboxylic acid or one of its salts at an acidic pH, or (d) of an anhydrous composition comprising at least one α-oxocarboxylic acid or one of its salts and of an aqueous composition comprising at least one sulfinic acid or one of its salts at an acidic or alkaline pH.

Preferably, according to the present invention, the composition for bleaching at an acidic pH is obtained by extemporaneous mixing at the time of use of two aqueous compositions, one of which comprises at least one sulfinic acid or one of its salts at an acidic or alkaline pH and the other of which comprises at least one α-oxocarboxylic acid or one of its salts at an acidic pH.

More particularly still, according to the present invention, the composition for bleaching at an acidic pH is obtained by extemporaneous mixing at the time of use of two aqueous compositions, one of which comprises at least sodium hydroxymethanesulfinate at an alkaline pH and the other of which comprises at least α-ketoglutaric acid at an acidic pH.

Depending on the bleaching process, at least one bleaching composition as defined above is applied to dyed fibers, at an application temperature between ambient temperature and 80° C., for a time sufficient to partially or completely destroy the coloring resulting from the oxidizing or nonoxidizing dyeing of keratinous fibers. The fibers are subsequently rinsed, or optionally washed with a shampoo, rinsed again and then dried. Following the post-shampooing rinsing, and preferably, a stage of neutralization of the reducing agent possibly remaining is carried out by applying, to the fibers, an aqueous hydrogen peroxide composition. The fibers are subsequently rinsed, shampooed, again rinsed and then dried.

The application temperature is preferably between ambient temperature and 60° C. and more preferably still between 35° C. and 50° C.

The time sufficient for the development of the bleaching of keratinous fibers is generally between 1 and 60 minutes and more specifically still between 5 and 30 minutes.

The examples which follow are intended to illustrate the invention without, however, limiting the scope thereof.

In these examples, the performance of the bleaching according to the invention was evaluated by measuring the bleaching ΔE of hair using a Minolta CM 2002 calorimeter in the CIE L*a*b* international system.

According to this system, the higher the value of L, the lighter the color or the less intense it is. Conversely, the lower the value of L, the darker the color or the more intense it is [L*=0 is black; L*=100 is white].
a* and b* indicate two axes of colors,
a* indicates the green/red color axis (+a* is red, −a* is green)) and
b* the blue/yellow color axis (+b* is yellow and −b* is blue).

Values for a* and b* of close to zero correspond to gray shades.

The bleaching ΔE is calculated by applying the following equation:

$$Δ E = \sqrt{{( {L}^{*} - {L}_{o}^{*} )}^{2} + {( {a}^{*} - {a}_{o}^{*} )}^{2} + {( {b}^{*} - {b}_{o}^{*} )}^{2}}$$

In this equation, ΔE represents the difference in color between two locks (in the present case, the bleaching), L*, a* and b* represent respectively the measurements of the bleached lock and L*, aₒ* and bₒ* representing respectively the measurements of the dyed lock.

The greater the value of ΔE, the greater the difference in color between the two locks and, in the present case, the greater the bleaching.

### EXAMPLE 1

Locks of hair were dyed beforehand with a commercial oxidation dye Movida 45, dark brown.

They were subsequently bleached by immersion for 20 minutes in the solutions of the following examples in a proportion of 10 g of solution per 1 g of treated hair. After rinsing with water, shampooing and drying, they were read with a calorimeter.

[A] The bleachings obtained from an aqueous solution (3) including the combination according to the invention were compared with two aqueous solutions (1) and (2) each including the compound of the prior art at a molar concentration at which it is present in the combination according to the invention:
(1) 0.0552 mol % of sodium hydroxymethanesulfinate dihydrate [8.5 weight %]
(2) 0.0068 mol % of a-ketoglutaric acid [1 weight %]
(3) 0.0552 mol % of sodium hydroxymethanesulfinate dihydrate [8.5 weight %]+0.0068 mol % of α-ketoglutaric acid [1 weight %],
   each of these solutions additionally including 0.25% by weight of the same surfactant (sodium olefinsulfonate) and having been adjusted before use to pH 2.7 with orthophosphoric acid and/or monoethanolamine.

Over an average of three dyed locks, the results were as follows:

| Solution | 1 | 2 | 3 |
|---|---|---|---|
| ΔE Mean | 4.8 | 2.1 | 11.4 |
| Standard deviation | 1.5 | 1.2 | 1.7 |

The results show that the solution (3), which corresponds to the combination according to the invention, is much greater with regard to bleaching (11.4) than the arithmetic sum of the bleachings of each of the compounds of the combination brought together (4.8+2.1=6.9).

[B] The bleachings obtained from an aqueous solution (3) including the combination according to the invention were compared with two aqueous solutions (5) and (6) each including the compound of the prior art at the molar concentration of the two compounds present in the combination according to the invention:
(5) 0.062 mol % (0.0552+0.0068 mol % ) of sodium hydroxymethanesulfinate dihydrate [9.56 weight %]
(6) 0.062 mol % (0.0552+0.0068 mol % ) of α-ketoglutaric acid [9.055 weight %]
(3) 0.0552 mol % of sodium hydroxymethanesulfinate dihydrate [8.5 weight %]+0.0068 mol % of α-ketoglutaric acid [1 weight %],
   each of these solutions additionally including 0.25% by weight of the same surfactant (sodium olefinsulfonate) and having been adjusted before use to pH 2.7 with orthophosphoric acid and/or monoethanolamine.

Over an average of three dyed locks, the results were as follows:

| Solution | 5 | 6 | 3 |
|---|---|---|---|
| ΔE Mean | 8.8 | 0.9 | 11.4 |
| Standard deviation | 0.5 | 1.0 | 1.7 |

The results show that the solution (3), which corresponds to the combination according to the invention, is synergistic with regard to bleaching since the bleaching brought about by this combination (ΔE=11.4) is much greater than the bleaching produced by each of the solutions [(5) ΔE=8.8] and [(6) ΔE=0.9] of the prior art each respectively including sodium hydroxymethanesulfinate dihydrate, for the solution (5), and α-ketoglutaric acid, for the solution (6), at the total molar concentration of these two compounds present in the combination.

### EXAMPLE 2

Locks of hair were dyed beforehand with a commercial oxidation dye Movida 45, dark brown, according to a protocol identical to that of example 1.

The aqueous solution (3) of example 1, including a combination according to the invention, was compared with an aqueous solution (4) including a combination of the prior art:
(4) 0.00552 mol % of ascorbic acid [9.715 weight %]+0.0068 mol % of a-ketoglutaric acid [1 weight %],
   each of these solutions additionally including 0.25% by weight of the same surfactant (sodium olefinsulfonate) and having been adjusted before use to pH 2.7 with orthophosphoric acid and/or monoethanolamine.

Over an average of three dyed locks, the results were as follows:

| Solution | 3 | 4 |
|---|---|---|
| ΔE Mean | 11.40 | 3.70 |
| Standard deviation | 1.70 | 0.75 |

These results show that the combination according to the invention bleaches much more (ΔE=11.40) than that of the prior art (ΔE=3.70).

### EXAMPLE 3

Locks of hair were dyed beforehand according to a protocol identical to that of example 1 with a commercial oxidation dye Majirouge Shade 6.66, to which were added 0.039% by weight of the cationic direct dye Basic Red 51 (dye of formula (IV2) and 0.035% by weight of the cationic direct dye Basic Yellow 87 (dye of formula (VI4).

They were subsequently bleached using the solutions (3) and (4) described in examples 1 and 2:
(3) 0.0552 mol % of sodium hydroxymethanesulfinate dihydrate [8.5 weight %]+0.0068 mol % of α-ketoglutaric acid [1 weight %],
(4) 0.00552 mol % of ascorbic acid [9.715 weight %]+0.0068 mol % of α-ketoglutaric acid [1 weight %].

Over an average of three dyed locks, the results were as follows:

| Solution | 3 | 4 |
|---|---|---|
| ΔE Mean | 43.9 | 21.3 |
| Standard deviation | 2.2 | 1.8 |

These results show that the combination according to the invention bleaches much more (ΔE=43.9) than that of the prior art (ΔE=21.3).

1. A composition for bleaching keratinous fibers dyed with oxidation dyes and/or direct dyes, in particular human keratinous fibers, such as the hair, comprising at least one dye reducing agent in an aqueous medium appropriate for bleaching at an acidic pH and characterized in that said reducing agent is a system combining (i) at least one sulfinic acid or one of their cosmetically acceptable salts with (ii) at least one α-oxocarboxylic acid or one of their cosmetically acceptable salts.

2. The composition as claimed in claim 1, characterized in that it exhibits a pH of between 1.8 and 6.

3. The composition as claimed in claims 1 or 2, characterized in that, among sulfinic acids and their salts, the choice is made of hydroxymethanesulfinic acid and its salts.

4. The composition as claimed in any one of the preceding claims, characterized in that the salt or salts of sulfinic acids are chosen from alkali metal sulfinates, alkaline earth metal sulfinates or zinc sulfinates.

5. The composition as claimed in claims 3 or 4, characterized in that it relates to sodium hydroxymethanesulfinate.

6. The composition as claimed in any one of the preceding claims, characterized in that the α-oxocarboxylic acid is α-ketoglutaric acid or one of [lacuna] alkali metal or alkaline earth metal salts.

7. The composition as claimed in any one of claims 1 to 5, characterized in that the α-oxocarboxylic acid is chosen from oxalic acid, glyoxalic acid, pyruvic acid or one of their alkali metal or alkaline earth metal salts.

8. The composition as claimed in any one of the preceding claims, characterized in that the sulfinic acid or acids or their cosmetically acceptable salts represent 0.01 to 20% by weight of the total weight of the composition.

9. The composition as claimed in claim 8, characterized in that they represent 0.1 to 10% by weight of the total weight of the composition.

10. The composition as claimed in any one of the preceding claims, characterized in that the α-oxocarboxylic acid or acids represent 0.01 to 15% by weight of the total weight of the composition.

11. The composition as claimed in claim 10, characterized in that they represent 0.1 to 10% by weight of the total weight of the composition.

12. The composition as claimed in any one of the preceding claims, characterized in that the ratio by weight of the sulfinic acid or acids or their salts to the α-oxocarboxylic acid or acids or their salts is between 10/1 and 1/10.

13. The composition as claimed in any one of the preceding claims, characterized in that the aqueous medium comprises one or more organic solvents present in a concentration ranging from 0.5 to 20% and preferably from 2 to 10% by weight with respect to the total weight of the composition.

14. The composition as claimed in any one of the preceding claims, characterized in that it comprises at least one thickening agent.

15. The composition as claimed in claim 14, characterized in that the thickening agent is a cellulose derivative, a guar derivative, a gum of microbial or plant origin or a synthetic thickener.

16. The composition as claimed in claims 14 or 15, characterized in that the thickening agent or agents represent 0.01 to 10% by weight of the total weight of the composition.

17. The composition as claimed in any one of the preceding claims, characterized in that it additionally comprises at least one basifying or acidifying agent in amounts ranging from 0.01 to 30% by weight of the total weight of the composition.

18. The composition as claimed in claim 17, characterized in that the basifying agent is chosen from ammonia, alkaline carbonates, alkanolamines, such as mono-, di- and triethanolamines and their derivatives, oxyethylenated and/or oxypropylenated ethylenediamines and hydroxyalkylamines, sodium hydroxide, potassium hydroxide and compounds of following formula (K): 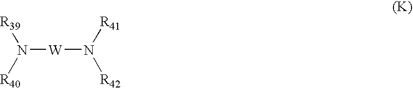
in which R is a propylene residue optionally substituted by a hydroxyl group or a C₁-C₄ alkyl radical and R₃₉, R₄₀, R₄₁, and R₄₂, which are identical or different, represent a hydrogen atom, a C₁-C₄ alkyl radical or a C₁-C₄ hydroxyalkyl radical.

19. The composition as claimed in claim 17, characterized in that the acidifying agent is chosen from inorganic or organic acids, such as etidronic acid, hydrochloric acid, orthophosphoric acid, carboxylic acids, such as tartaric acid, citric acid or lactic acid, or sulfonic acids.

20. The composition as claimed in any one of the preceding claims, characterized in that it is obtained by extemporaneous mixing at the time of use either (a) of an anhydrous composition comprising at least one bleaching system as defined in any one of the preceding claims and of an aqueous composition at an acidic pH, or (b) of two aqueous compositions, one of which comprises at least one sulfinic acid or one of its salts at an acidic or alkaline pH and the other of which comprises at least one α-oxycarboxylic acid or one of its salts at an acidic pH, or (c) of an anhydrous composition comprising at least one sulfinic acid or one of its salts and of an aqueous composition comprising at least one α-oxocarboxylic acid or one of its salts at an acidic pH, or (d) of an anhydrous composition comprising at least one α-oxocarboxylic acid or one of its salts and of an aqueous composition comprising at least one sulfinic acid or one of its salts at an acidic or alkaline pH.

21. The composition as claimed in claim 20, characterized in that it is obtained by extemporaneous mixing at the time of use of two aqueous compositions, one of which comprises at least one sulfinic acid or one of its salts at an acidic or alkaline pH and the other of which comprises at least one α-oxocarboxylic acid or one of its salts at an acidic pH.

22. The composition as claimed in claim 21, characterized in that it is obtained by extemporaneous mixing at the time of use of two aqueous compositions, one of which comprises at least sodium hydroxymethanesulfinate at an alkaline pH and the other of which comprises at least x-ketoglutaric acid at an acidic pH.

23. The composition as claimed in any one of the preceding claims, characterized in that it is provided in the form of a gel, of a cream, of a mousse, of a solution or of an emulsion.

24. A multicompartment device intended (i) for the dyeing and then (ii) for the bleaching of keratinous fibers dyed with oxidation dyes and/or direct dyes, in particular human keratinous fibers, such as the hair, characterized in that it comprises a first compartment comprising a composition for the oxidizing dyeing or the nonoxidizing dyeing of said fibers and, in a second compartment, the composition for the reductive bleaching of said colored fibers as defined in any one of claims 1 to 23.

25. The device as claimed in claim 24, characterized in that, in the first compartment, the composition for oxidizing dyeing comprises oxidation bases and/or couplers which form an oxidation dye by mixing with an oxidizing agent.

26. The device as claimed in claim 25, characterized in that, in the first compartment, the composition for oxidizing dyeing additionally comprises direct dyes.

27. The device as claimed in claim 24, characterized in that, in the first compartment, the composition for oxidizing dyeing comprises direct dyes, preferably cationic direct dyes, which form a lightening direct dye by mixing with an oxidizing agent.

28. The device as claimed in claim 24, characterized in that, in the first compartment, the composition for nonoxidizing dyeing comprises direct dyes and preferably cationic direct dyes.

29. The device as claimed in claims 27 or 28, characterized in that the cationic direct dyes are chosen from the dyes with the following structures (I) to (VII) and their mesomeric forms:
(i) dyes of formulae (I), (II)a, (II)b, (III)a and (III)b: 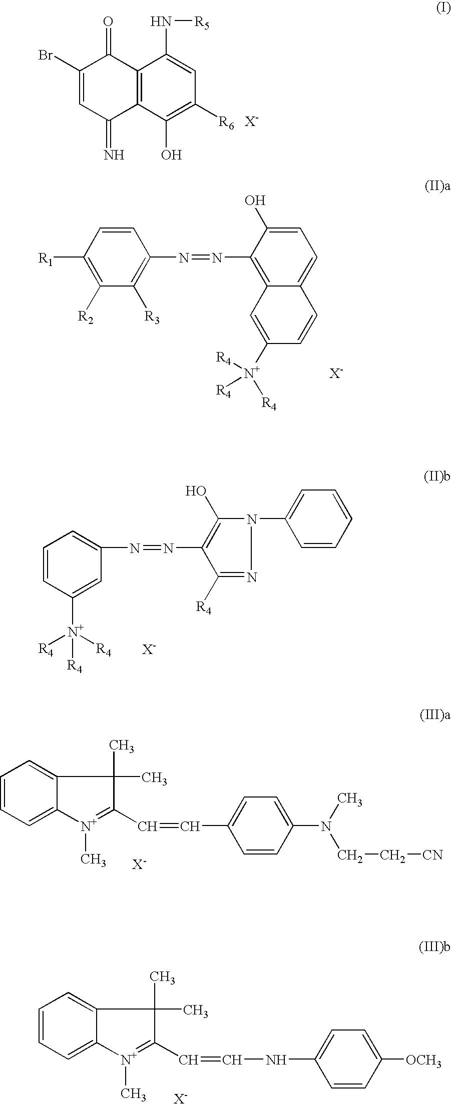
in which formulae (I), (II)a, (II)b, (III)a and (III)b:
R₁ represents a hydrogen atom or an amino radical;
R₂ represents a hydrogen atom or a nitro group;
R₃ represents a hydrogen atom, a nitro group or a C₁-C₄ alkoxy radical;
R₄ represents a C₁-C₄ alkyl radical;
R₅ represents a hydrogen atom or a para-triC₁-C₄alkylammoniophenyl group;
R₆ represents a bromine atom or an NH-para-triC₁-C₄alkylammoniophenyl group;
X⁻ represents an anion;
(ii) dyes of formulae (IV), (V), (VI), (VI′) and (VII):
a) the compounds of following formula (IV): 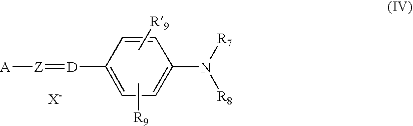
in which:
Z and D represent, which are identical or different, a nitrogen atom or the —CH—radical,
R₇ and R₈, which are identical or different, represent a hydrogen atom; a C₁-C₄ alkyl radical which can be substituted by a —CN, —OH or —NH₂ radical or form, with a carbon atom of the benzene ring, an optionally oxygen-comprising or nitrogen-comprising heterocycle which can be substituted by one or more C₁-C₄ alkyl radicals; or a 4′-aminophenyl radical,
R₉ and R′₉, which are identical or different, represent a hydrogen atom, a halogen atom, a cyano radical, a C₁-C₄ alkyl radical, a C₁-C₄ alkoxy radical or an acetyloxy radical,
X⁻ represents an anion;
A represents a group chosen by the following structures A1 to A19: 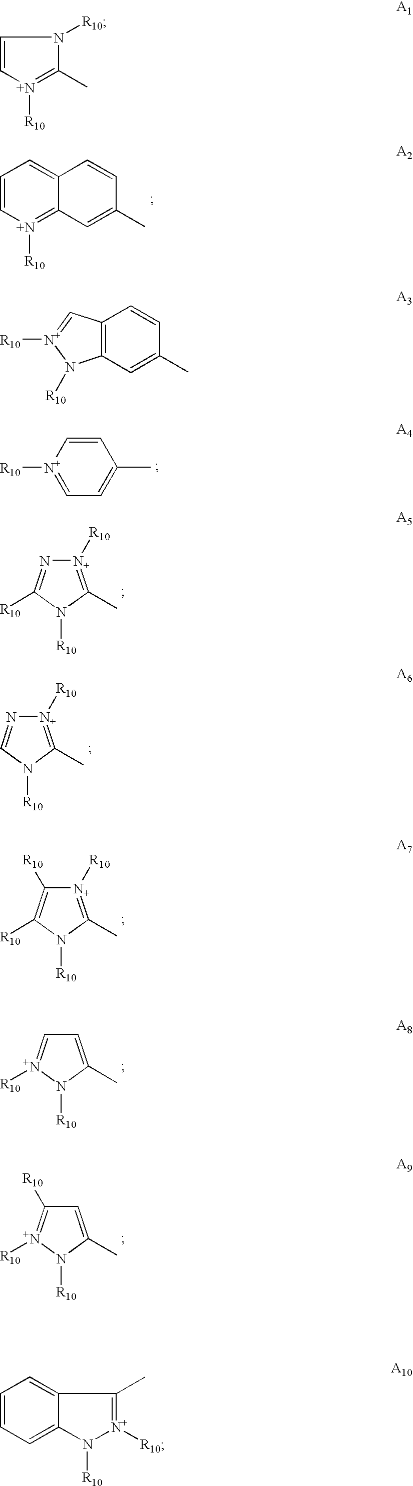 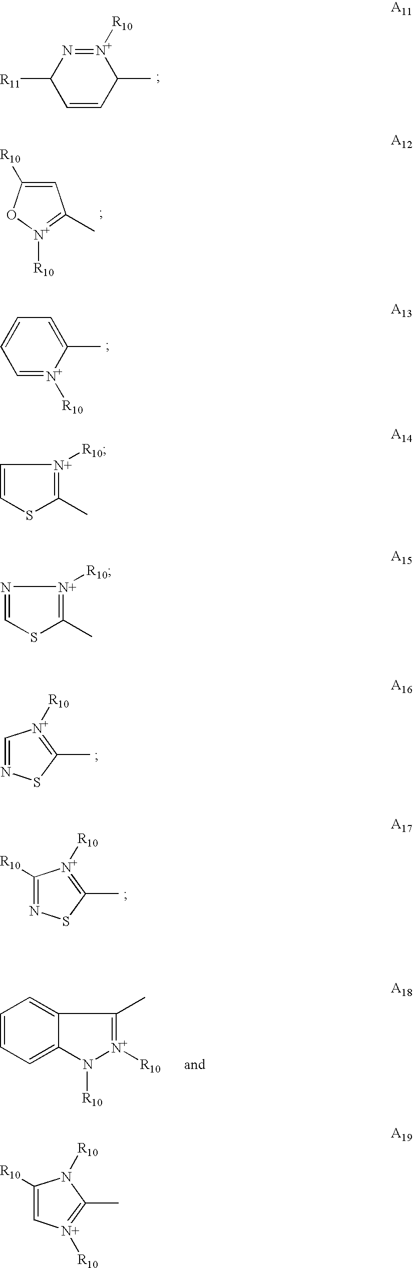
in which R₁₀ represents a C₁-C₄ alkyl radical which can be substituted by a hydroxyl radical and R₁₁ represents a C₁-C₄ alkoxy radical;
b) the compounds of following formula (V): 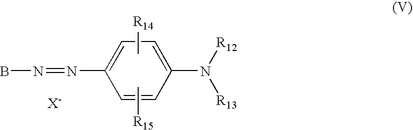
in which:
R₁₂ represents a hydrogen atom or a C₁-C₄ alkyl radical,
R₁₃ represents a hydrogen atom, an alkyl radical which can be substituted by a —CN radical or by an amino group, or a 4′-aminophenyl radical or forms, with R₁₂, an optionally oxygen-comprising and/or nitrogen-comprising heterocycle which can be substituted by a C₁-C₄ alkyl radical,
R₁₄ and R₁₅, which are identical or different, represent a hydrogen atom, a halogen atom, a C₁-C₄ alkyl radical, a C₁-C₄ alkoxy radical or a —CN radical,
X⁻ represents an anion;
B represents a group chosen by the following structures B1 to B6: 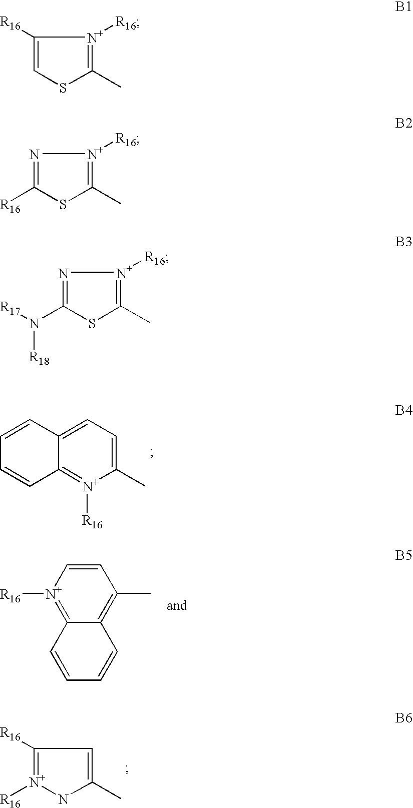
in which R₁₆ represents a C₁-C₄ alkyl radical and R₁₇ and R₁₈, which are identical or different, represent a hydrogen atom or a C₁-C₄ alkyl radical;
c) the compounds of following formulae (VI) and (VI′): 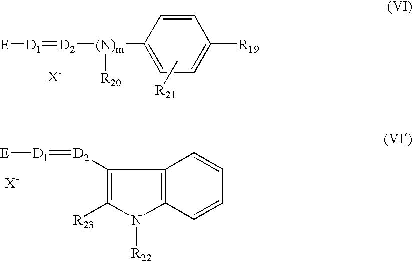
in which:
R₁₉ represents a hydrogen atom, a C₁-C₄ alkoxy radical, a halogen atom, such as bromine, chlorine, iodine or fluorine, or an amino radical,
R₂₀ represents a hydrogen atom or a C₁-C₄ alkyl radical or forms, with a carbon atom of the benzene ring, a heterocycle which optionally comprises oxygen and/or is substituted by one or more C₁-C₄ alkyl groups,
R₂₁ represents a hydrogen atom or a halogen atom,
R₂₂ and R₂₃, which are identical or different, represent a hydrogen atom or a C₁-C₄ alkyl radical,
D₁ and D₂, which are identical or different, represent a nitrogen atom or the —CH group,
m =0 or 1,
X⁻ represents an anion;
E represents a group chosen by the following structures E1 to E8: 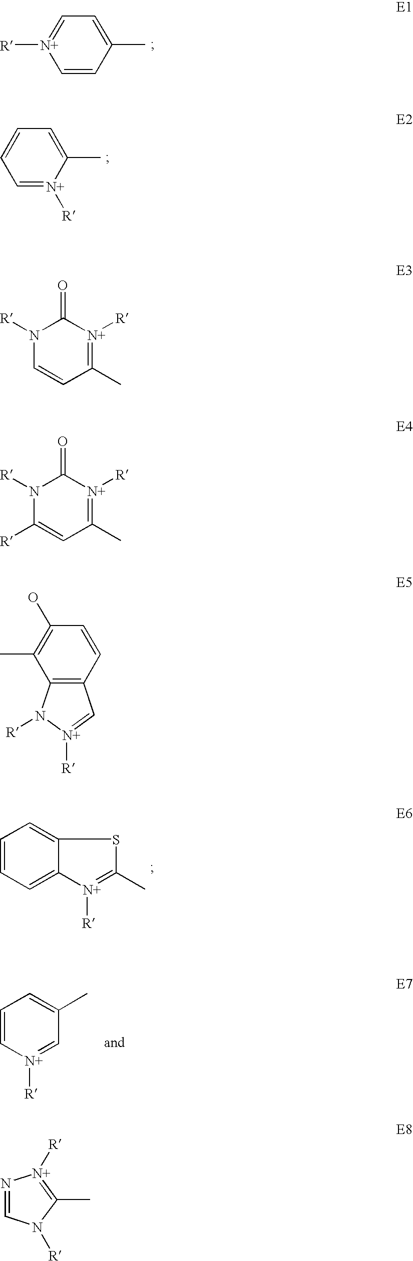
in which R′ represents a C₁-C₄ alkyl radical;
when m=0 and when D₁₃ represents a nitrogen atom, then E can also denote a group with the following structure E9: 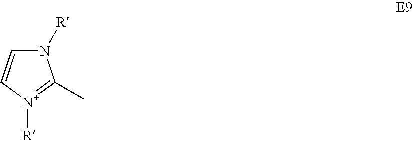
in which R′represents a C₁-C₄ alkyl radical.
d) the compounds of following formula (VII):
G—N═N—J (VII)
in which:
the symbol G represents a group chosen from the following structures G₁ to G₃: 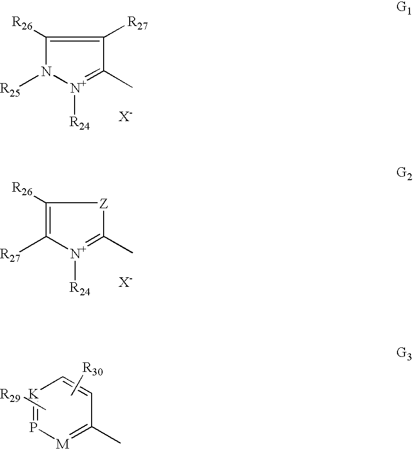
in which structures G₁ to G₃,
R₂₄ denotes a C₁-C₄ alkyl radical or a phenyl radical which can be substituted by a C₁-C₄ alkyl radical or a halogen atom chosen from chlorine, bromine, iodine and fluorine;
R₂₅ denotes a C₁-C₄ alkyl radical or a phenyl radical;
R₂₆ and R₂₇, which are identical or different, represent a C₁-C₄ alkyl radical or a phenyl radical or together form, in G₁, a benzene ring substituted by one or more C₁-C₄ alkyl, C₁-C₄ alkoxy or NO₂ radicals or together form, in G₂, a benzene ring optionally substituted by one or more C₁-C₄ alkyl, C₁-C₄ alkoxy or NO₂ radicals;
R₂₆ can additionally denote a hydrogen atom;
Z denotes an oxygen or sulfur atom or an —NR₂₅ group;
M represents a —CH, —CR (R denoting C₁-C₄ alkyl) or —NR₂₈(X⁻)ᵣ group;
K represents a —CH, —CR (R denoting C₁-C₄ alkyl) or —NR₂₈(X⁻)ᵣ group;
P represents a —CH, —CR (R denoting C₁-C₄ alkyl) or —NR₂₈(X⁻)ᵣ group; r denotes zero or 1;
R₂₈ represents an O⁻ atom, a C₁-C₄ alkoxy radical or a C₁-C₄ alkyl radical;
R₂₉ and R₃₀, which are identical or different, represent a hydrogen atom, a halogen atom, a C₁-C₄ alkyl radical, a C₁-C₄ alkoxy radical or an —NO₂ radical;
X⁻ represents an anion;
the symbol J represents:
(a) a group with the following structure J₁: 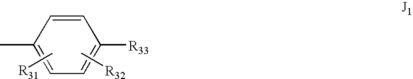
in which structure J₁,
R₃₁ represents a hydrogen atom, a halogen atom, a C₁-C₄ alkyl radical, a C₁-C₄ alkoxy radical or an —OH, —NO₂, —NHR₃₄, —NR₃₅R₃₆ or —NHCO(C₁-C₄)alkyl radical or forms, with R₃₂, a 5- or 6-membered ring which may or may not comprise one or more heteroatoms chosen from nitrogen, oxygen or sulfur;
R₃₂ represents a hydrogen atom, a halogen atom, a C₁-C₄ alkyl radical or a C₁-C₄ alkoxy radical
or forms, with R₃₃ or R₃₄, a 5- or 6-membered ring which may or may not comprise one or more heteroatoms chosen from nitrogen, oxygen or sulfur;
R₃₃ represents a hydrogen atom, an —OH radical, an —NHR₃₄ radical or an —NR₃₅R₃₆ radical;
R₃₄ represents a hydrogen atom, a C₁-C₄ alkyl radical, a C₁-C₄ monohydroxyalkyl radical, a C₂-C₄ polyhydroxyalkyl radical or a phenyl radical;
R₃₅ and R₃₆, which are identical or different, represent a C,-C₄ alkyl radical, a C₁-C₄ monohydroxyalkyl radical or a C₂-C₄ polyhydroxyalkyl radical;
(b) a 5- or 6-membered nitrogenous heterocyclic group which can include other heteroatoms and/or carbonyl groups and which can be substituted by one or more C₁-C₄alkyl, amino or phenyl radicals,
and in particular a group with the following structure J₂: 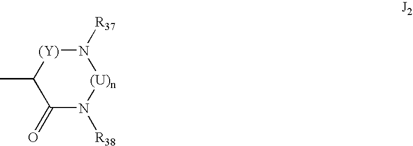
in which structure J₂,
R₃₇ and R₃₈, which are identical or different, represent a hydrogen atom, a C₁₃-C₁₀ alkyl radical or a phenyl radical;
Y denotes the —CO— radical or the 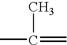
radical;
n=0 or 1, with, when n denotes 1, U denotes the —CO— radical,
and the mesomeric forms of these structures (IV) to (VII).

30. A process for bleaching keratinous fibers dyed with oxidation dyes and/or direct dyes, in particular human keratinous fibers, such as the hair, characterized in that the composition as defined in any one of claims 1 to 23 is applied to said fibers, at an application temperature between ambient temperature and 80° C., for a time sufficient to partially or completely destroy the dyeing of the hair, the fibers are subsequently rinsed, they are optionally washed with a shampoo, they are rinsed again and then they are dried.

31. The process as claimed in claim 30, characterized in that the application temperature is between 35° C. and 50° C.

32. The process as claimed in claim 30 or 31, characterized in that the time sufficient for the development of the bleaching is between 1 and 60 minutes.

33. The process as claimed in claim 32, characterized in that the time sufficient for the development of the bleaching is between 5 and 30 minutes.

34. The process as claimed in any one of claims 30 to 33, characterized in that, after the rinsing following the shampooing, an aqueous hydrogen peroxide composition is applied to the fibers, they are rinsed, they are washed with a shampoo, they are again rinsed and they are dried.
